(19) United States
(12) Patent Application Publication
(10) Pub. No.: US 2021/0201690 A1
(43) Pub. Date: Jul. 1, 2021

(54) LEARNING MANAGEMENT SYSTEM

(71) Applicants: Tan Boon Keat, Singapore (SG); Loh Yin Huei, Singapore (SG); Tan Yi Kai, Singapore (SG); Tan Yi Kheng, Singapore (SG)

(72) Inventors: Tan Boon Keat, Singapore (SG); Loh Yin Huei, Singapore (SG); Tan Yi Kai, Singapore (SG); Tan Yi Kheng, Singapore (SG)

(21) Appl. No.: 17/137,065

(22) Filed: Dec. 29, 2020

(30) Foreign Application Priority Data
Dec. 31, 2019 (SG) 10201914016Q
Dec. 29, 2020 (SG) 10202013173Q

(51) Int. Cl. G09B 19/00; G09B 7/00; G16H 40/63

(52) U.S. Cl. CPC G09B 19/00; G16H 40/63; G09B 7/00

(57) ABSTRACT

A learning management system (LMS) for assisting a student to prepare for an examination based on time-estimates is presented. The LMS receives inputs from the students about available time to study. The study plan includes an estimation of time needed to complete the entire syllabus of the examination. The LMS includes a record of time-estimates needed to complete the study material and maintain a study plan. In addition, the LMS maintain a difficulty-level estimates for each of the study materials as well as the proficiency level of the students. The LMS is adapted to generate a study plan for the student and adaptively update the study plan.

### CROSS REFERENCE TO RELATED APPLICATIONS

This application claims the benefit of Singapore Patent Application No. 10202013173Q filed Dec. 29, 2020, and Singapore Patent Application No. 10201914016Q filed Dec. 31, 2019, the disclosure of each of which are incorporated herein by reference.

### BACKGROUND

The present invention relates to a learning management system (referred herein after as LMS), which is designed to assist a student whose objective is to learn at least one subject within a targeted study period.

Education often involves assessment. Assessments are done primarily through examination. Everyone has experience taking a major examination at various stages of his or her studies. Some examinations may be more critical as compared to other examinations. For example, in most countries, a final year examination at the end of primary school or secondary school may be more critical. Students often spend a significant amount of time preparing for the examination. As the scope of such final year examinations may be broader than a school term examination, preparation for such final year examinations may be more difficult as compared to an ordinary examination. Often, students may not prepare for examination well and end up taking the final year examination without completing whatever he or she needs to prepare. Burning midnight oil to rush through the last-minute preparation is not uncommon for students. For term-end examination that takes place within a few weeks or a few months, the problem may be less severe but students, especially those without discipline to study consistently, may also struggle to complete the preparation in a timely manner.

In addition to the time management issues described above, another issue with examination preparation is related to the selection of the study materials. Generally, a student blindly purchases study materials, for example, worksheets or workbooks. The students often complete the study materials without knowing his strength and his weakness. For example, in Singapore, a student preparing for the Primary School Leaving Examination (PSLE) often does practices on a large number of past examination papers. The problem with this approach is that the student may spend most of the time on known questions or topics. Repetition, such as redoing questions answered incorrectly or questions on weaker topics, is often neglected because there is no such tracking or goals. Most students are without a study plan, in which they know they can complete within a targeted study period. Often, students buy assessment books but ended up with a substantial portion untouched because the students later find out that they do not have time. In addition, due to the reason that assessment books are pre-prepared without considering the needs of individual students, many students ended up not completing the assessment books because they later find out that the assessment books are not at a suitable level. Those who complete the assessment books may also find out that they wasted too much time on something that they already know.

Psychologist K. Anders Ericsson is one of the leading scholars who had been actively studying the science behind why some people have extraordinary abilities or expert performance. Ericsson and his fellow researchers have introduced the concept of deliberate practice, or a way for anyone to grow his expertise through a series of planned action steps, reflections, and collaboration. Involved in the Deliberate Practice Plan are: (a) setting goals, (b) focused practice, (c) focused feedback, (d) observing and discussing teaching, and (e) monitoring progress. Applying the concept of deliberate practice for a specific area, such as learning piano with a coach, may be more straightforward as compared to preparing an examination for multiple subjects with a broad syllabus. A good teacher may guide the student well through his or her experience, but it would be impossible for the teacher to understand every aspect of the student, as the teacher is often tasked with a large number of students. In order for the teachers to do a better job, it is always best for the teachers to have some clarity as to the strength of each individual student. Using the traditional classroom type teaching methods, the strength of each individual student will not be known until the teachers spend more than one semester with the student. Unfortunately, most teachers do not teach the same students for more than two semesters.

Preparing for examinations can be stress free and can be more efficient if the above shortcomings can be addressed. As an examination is a form of assessment of learning, therefore, in other words, the efficiency in learning can be improved.

### BRIEF DESCRIPTION OF THE DRAWINGS

Illustrative embodiments by way of examples, not by way of limitation, are illustrated in the drawings. Throughout the description and drawings, similar reference numbers may be used to identify similar elements. The drawings may be simplified illustrative views rather than precise engineering drawings. The drawings are for illustrative purpose to assist understanding and may not necessarily be drawn per actual scale.

FIG. 1 shows an illustrative block diagram of an LMS having a scheduling management system;

FIG. 2A shows an illustrative example of the target planning interface;

FIG. 2B shows an example of an electronic based study material selection interface;

FIG. 2C shows an illustrative example of a schedule planning interface;

FIG. 2D shows an additional planning chart;

FIG. 2E shows an illustration of a progress report that forms a part of the planning interface that is suitable for implementation using a paper-based planner;

FIG. 2F shows an illustrative example of the progress report generator interface;

FIG. 2G shows an illustrative example of a progress monitoring interface of a paper-based LMS;

FIGS. 3A-3C shows a method for an LMS illustrated in previous embodiments;

FIG. 4 shows an illustrative block diagram of instruction sets of an LMS for implementing various methods illustrated in subsequent embodiments;

FIG. 5 shows a method for managing and monitoring a learning plan;

FIG. 6 shows a method for providing a customized study content;

FIG. 7 shows a method utilizing prior use data to improve efficiency of a study plan;

FIG. 8 shows a method for generating a solution tutorial;

FIG. 9 shows examples of similar questions;

FIG. 10 shows a method for providing education materials;

FIGS. 11A-11C show evaluation infographic reports regarding examination readiness of a student; and

FIG. 12 shows a method for generating the evaluation infographic report.

### DETAILED DESCRIPTION

### Scheduling Management System

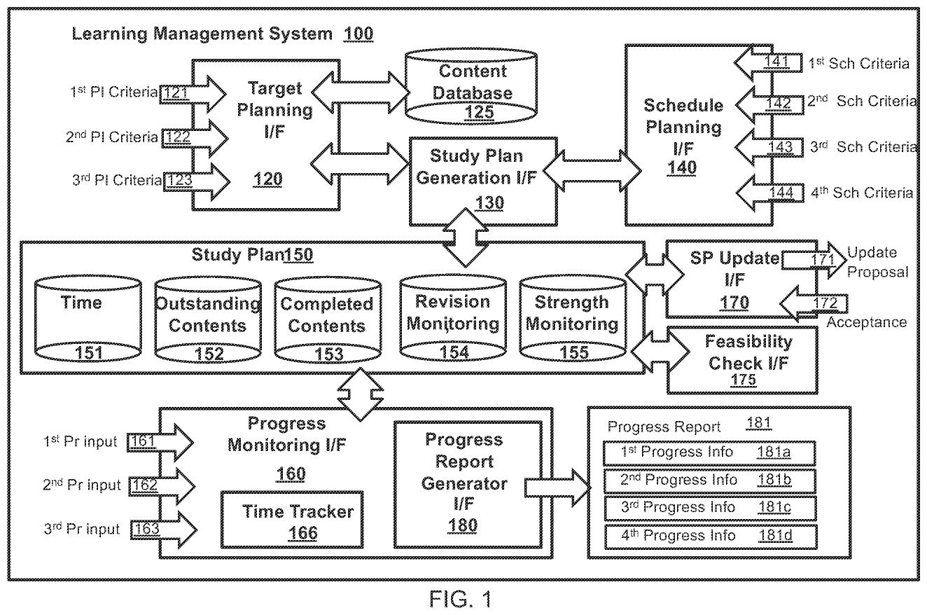

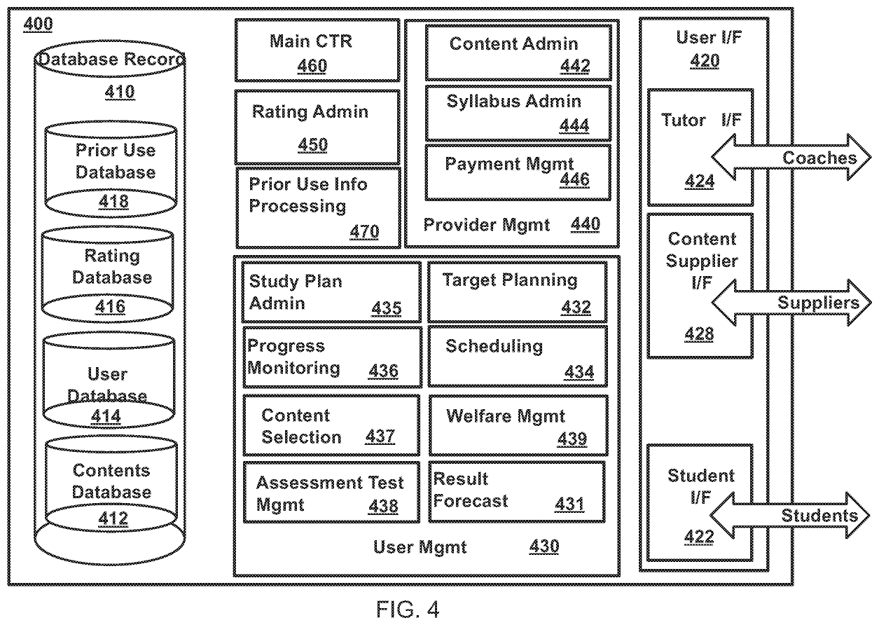

FIG. 1 shows an illustrative block diagram of an LMS 100, which comprises a target planning interface 120, a schedule planning interface 140, a study plan generation interface 130, a progress monitoring interface 160 and a progress report generator interface 180. In one embodiment where the LMS 100 comprises a computerized system such as a computer, smart phone, or any other computing devices, each of the interfaces may be instructions executable by a computer system for interactions with a student or other computation means. In another embodiment where LMS 100 comprises a paper-based planner, the LMS 100 may be a page of the planner for interacting with a student. The LMS 100, the LMS 400 (see FIG. 4) and other LMS described in this specification are provided for a human subject, also referred to as a student throughout the specification and the claims. In addition to the human subject, the LMS 100, LMS 400 and other LMS are provided to additional students. For avoidance of doubt, unless specified otherwise, the term “a plurality of students” include the specific human subject, as well as other students who use the LMS. The LMS 100 may optionally comprise a content database 125, a study plan 150, a study plan update interface 170 and other interfaces as necessary. The study plan 150 comprises an estimation of study material and an estimation of time needed to complete the study material. For example, the study plan 150 may include a time schedule as to when the student is going to do what, the materials that the student needs to go through and other plans related to the plan that the student is going to execute in order to sit for the examination or achieve a learning outcome. In one embodiment, LMS may shortlist an identified set of study materials selected from the study material database into the study plan. Each of the identified set of study materials comprise a time-estimate. The LMS then adaptively select material from the shortlisted identified set of study materials.

The target planning interface 120 is configured to receive a first planning criterion 121, and a second planning criterion 122. The target planning interface 120 may be optionally configured to receive a third planning criterion 123 or an additional planning criterion. Referring to FIG. 1, the first planning criterion 121 comprises at least one subject targeted by a student. The first planning criterion may comprise a plurality of subjects. If a student is going to sit for an examination, the first planning criterion 121 may comprise all or a portion of the subjects the student is going to be examined that require preparation and a lot of studies. The second planning criterion 122 comprises at least one study material corresponding to the at least one subject. The at least one study material comprises study materials such as revision books, worksheets, sets of assessment questions, past examination papers, programs corresponding to each of the subjects that the student is going to sit for the examination, and any other materials that the student may read, listen, practice or watch to prepare for the examination. The optional third criterion 123 may comprise an additional or optional study material corresponding to the at least one subject to be used conditionally. For illustration purposes, consider a scenario of a student, Mike, who will be taking an examination within one-year period in the subjects of Science and Mathematics. Mike plans to use workbooks titled Weekly Science MCQ, and Advanced Science Workbook as preparation for Science. In addition, Mike plans to use workbooks titled Weekly Math Drill, and Challenging Math Workbook as preparation for Mathematics. The first planning criterion 121 is related to the subjects, which are Science and Mathematics. The second planning criterion 122 is related to the study material, i.e. (1) Weekly Science MCQ and Advanced Science Workbook for Science; (2) Weekly Math Drill and Challenging Math Workbook for Mathematics.

The schedule planning interface 140 is configured to receive at least a first schedule criterion 141, and a second schedule criterion 142. The first schedule criterion 141 comprises a targeted study period allocated for learning. The targeted study period allocated for learning comprises a time period from a start day the student uses the LMS 100 towards a date before the student taking a major examination. A major examination is an examination that is more important to the student as compared to other examinations. The second schedule criterion 142 comprises a duration estimate for the student to complete the at least one study material, or duration estimates for each of the at least one study material. In Mike's case as an example, his first schedule criterion 141 is the period one year allocated for studying. For example, the schedule planning interface 140 is configured to guide Mike, the student, to provide an estimate for the duration needed to complete each chapter of the study material of Weekly Science MCQ as well as Advance Science Workbook. Such estimates of the duration may be neglected by students. One reason is that the information needed are not provided by any prior system, or even if provided, the estimates are inaccurate. The reason is that even for one subject and for one study material, the estimates are difficult to be provided for an obvious reason that the student has not even seen the contents. The schedule planning interface 140 is configured to guide the student based on prior use data by other students which are grouped together in accordance with the strength in the subject, and/or other age group, location or other similar background criteria which will be further explained in subsequent embodiments. For this reason, the estimates provide through the schedule planning interface 140 are more accurate. As explained further, the estimates can be further improved as the student continues to use the system 100.

The target planning interface 120 may be optionally configured to guide the student to determine the at least one study material from a content database 125 provided digitally through a website or an app for mobile phone. A plurality of study materials are listed with statistical information based on prior use by other students. For example, the statistic may show the popularity of the contents as to how many students have used or recommended the material, and how a specific group of students did in a specific study material. The specific group may be selectable by the student in accordance with background criteria such as assessment results, age group, geometrical location or other related information. The statistic information may include the time needed to complete each chapter of the at least one study material based on the prior use data by other students. The target planning interface 120 may be configured to provide a filtering function interface so as to enable the student to see how much time is needed by a specific group of students. For example, the target planning interface 120 may be configured to show the study materials used by the top 10% high score students. In this way, a student who remain in the top 10% range will find the information more applicable and more accurate. Without the prior use data or estimate as discussed above regarding the second planning criterion 142, most students tend to overestimate or underestimate their ability resulting in last minute rush, or inability to complete the study plan prior to an end date of the targeted study period, usually the final examination date!

The schedule planning interface 140 may be adapted to receive a third schedule criterion 143 and a fourth schedule criterion 144. In FIG. 1, the third schedule criterion 143 may be an estimate of time available for learning within a predetermined short period, while the fourth schedule criterion 144 may be an estimate of time allocated for doing revision. This may be better understood considering Mike's case explained earlier. Mike may estimate 20 hours available for learning per week, which forms the third schedule criterion 143. Mike may estimate 30 hours of revision for the subject of Science and 40 hours of revision for the subject of Mathematics, which would form the fourth schedule criterion 144. The first, second, third and fourth schedule criteria 141-144 may be used to generate a study plan 150 together with the feasibility of the plan. At an initial stage, the second, third and fourth schedule criteria 142-144 may not be precise. The schedule planning interface 140 may be configured to improve the accuracy of the second, third and fourth schedule criteria 142-144 as the student starts to use the system and provide progress information. For this reason, the first, second, third and fourth schedule criteria 141-144 may be adaptively updated and adjusted from time to time. In a similar manner, the study plan can be adjusted accordingly. Going back to the example of Mike, Mike's wish is to study 20 hours per week. That means a total of 1040 hours per year. As time goes by, the learning system 100 may be able to compute that an improvement is needed. Mike will be then be prompted by the study plan update interface 170 to choose a new recommended third schedule criterion 143, or to adjust the third schedule criterion 143 accordingly. For example, if one month later, it is found out that Mike is only able to study 16 hours per week on average, the study plan update interface 170 of the LMS 100 may prompt him to change the value of the second planning criterion 142 accordingly. The learning assistance system will then adaptively update the study plan 150, and/or perform a feasibility check.

The progress monitoring interface 160 is configured to receive a first progress input 161 comprising a subset of the at least one study material completed (completed contents) within a predetermined short period. The progress monitoring interface 160 may be adapted to receive a second progress input 162 comprising information related to an amount of revision done, and a third progress input 163 comprising information related to a plurality of test scores or other assessment results of the at least one subject. The inputs may be performed by the student or may be initiated or suggested by the progress monitoring interface 160. Assume that Mike has completed chapter 1 of both Weekly Science MCQ and Advance Science Workbook in a month. He has also done revision for this chapter. In this case, he would provide the first progress input 161 as chapter 1 of Weekly Science MCQ and Advance Science Workbook. He would indicate that the revision of chapter 1 has been done with regards to the second progress input 162. The test scores he obtained for Chapter 1 in Weekly Science MCQ and Chapter 1 in Advance Science Workbook would form the third progress input 163. The progress monitoring interface 160 may comprise a time tracker 166 that is configured to compute a current time and determine a percentage of time that has passed by as compared to the remaining time towards the targeted study period.

The study plan generation interface 130 is configured to compute, or to facilitate the computation of a study plan 150 based on the first planning criterion 121, the second planning criterion 122, the first schedule criterion 141 and the second schedule criterion 142. The LMS 100 may additionally comprise a feasibility check interface 175 to provide an indication of feasibility of study plan 150. Optionally, the third planning criterion 123, the third and fourth schedule criteria 143-144 may be considered to generate the study plan 150 as well as the feasibility check by the feasibility check interface 175. Based on the information of subject, study material, targeted study period and duration needed for each material, the study plan 150 may be computed for the student to follow through and stay on track. In case there is not enough time for the student to complete the materials, the feasibility check interface 175 may comprise an interface to inform the student to make necessary changes, such as reducing the study material, lengthening the targeted study period for study or shortening the duration needed for each material. The study plan 150 may comprise a time component 151. The time component 151 comprises a plurality of information related to the time related data based on the planning criteria 121-123, the schedule criteria 141-144 and the progress input 161-163. For example, the time component may comprise a first information indicating the total remaining time towards the targeted study period, and a second time information indicating the amount of time used for studying within the targeted study period, and a third time information indicating the current time stamp. The study plan 150 may also comprise outstanding content component 152 and a completed content component 153. The outstanding content component 152 indicates a portion of at least one study material yet to be studied by the student while the completed content component 153 indicates a portion of the at least one study material completed by the student. The study plan 150 may further comprise a revision monitoring component 154 and a strength monitoring component 155. The revision monitoring component 154 comprises information such as an amount of time spent on doing revision, portion of the at least one study material that has been revised, or any other information related to doing revision. The strength monitoring component 155 comprises at least a test score of the at least one subject, or any other information related to how the student performed in the at least one subject.

The progress report generator interface 180 interactively generates a progress report 181 to provide a first progress information 181a that is indicative of an amount or a ratio estimate of the at least one study material completed by the student relative to the study plan 150. The progress report generator interface 180 may be adapted to generate a second progress information 181b, which illustrates an estimate or percentage estimate of revision done within the predetermined short period, or within an intermediate period longer than the predetermined short period but shorter than the targeted study period. The progress report generator interface 180 may be adapted to generate a third progress information 181c and a fourth progress information 181d. The third progress information 181c indicates a comparison between a percentage of the amount of the at least one study material completed by the student, and a percentage of time that has passed by as compared to a remaining time towards the targeted study period. The fourth progress information 181d indicates a comparison of the first progress information 181a of the student and a corresponding first progress information 181a of a different student or a group of students. The third progress information 181c and the fourth progress information 181d will provide a further detailed indication to the student whether he is on track towards the study plan that will not be available otherwise.

The study plan update interface 170 may be used for computing a study plan improvement proposal 171 based on the first progress information 181a. The study plan update interface 170 may allow a student to make changes to a study plan improvement proposal 171 to form the accepted study plan improvement proposal 172. Consider the case where Mike, from his progress report, realizes that he needs to speed up his study in order to complete all the study materials within the targeted study period. The study plan improvement proposal 171 may suggest to Mike to reduce the study material (the second planning criterion 122) or to shorten the duration needed for the material (the second schedule criterion 142). Mike may decide to keep all the study materials but shorten the duration needed for some of the materials. He may also make an effort to increase his study time (the third schedule criterion 143). Optionally, the study plan 150 may be updated adaptively through the information provided in the target planning interface 120. The target planning interface 120 may be configured to receive a third planning criterion 123 or other optional planning criterion. The third planning criterion 123 may comprise an additional study material corresponding to the at least one subject. The study plan generation interface 130 is configured to adaptively add the additional study material into the study plan 150 only if a predetermined milestone is achieved. For example, a student's progress may be ahead of the study plan 150. In that scenario, the time required to complete the outstanding contents 152 may become way less compared to the remaining duration towards the targeted study period. Therefore, the target planning interface 120 may trigger the student to add in the third planning criterion 123 so that the additional study material will be incorporated into the study plan 150. Refer back to the example of Mike. Assume that Mike has completed half of the study materials by the end of the 3 months, which he had originally planned to complete in 6 months. The time needed to complete the outstanding contents, per original plan, is 6 months. Mike is ahead of schedule and he has 67% of the remaining period 9 months to complete the study material. Since there is additional time, the learning assistance system 100, through the study plan update interface 170, may prompt Mike to consider adding more study materials that is used by students of a higher group to the plan. The learning assistance system 100 may be configured to check whether Mike's progress is ahead of the study plan 150, either adaptively when a progress is made or periodically, or at any time when Mike made an input. The target planning interface 120 may be triggered to ask for the third planning criterion 123 when the student has progressed faster than expected rate or has exceeded a specific target.

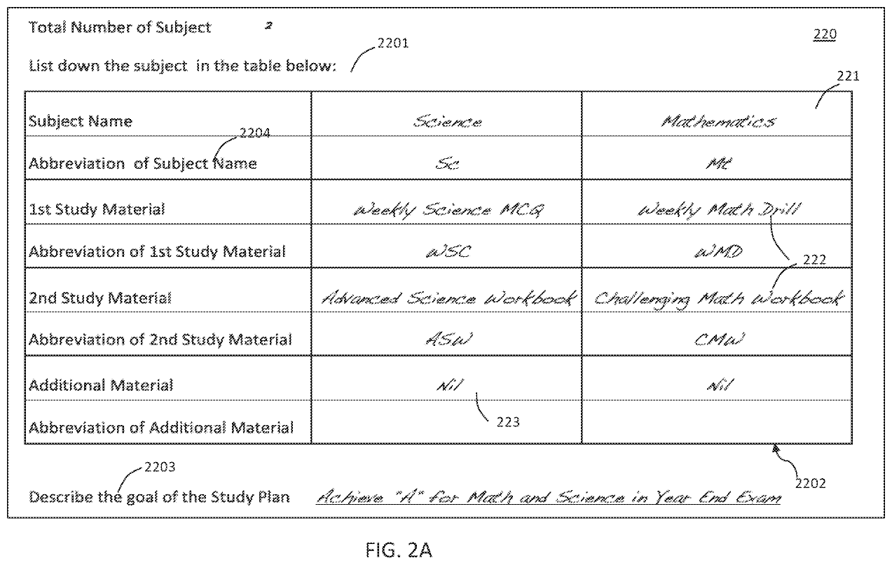

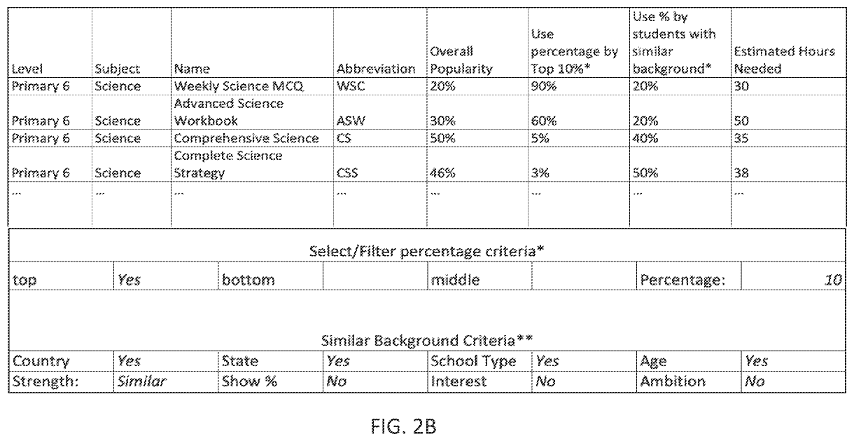

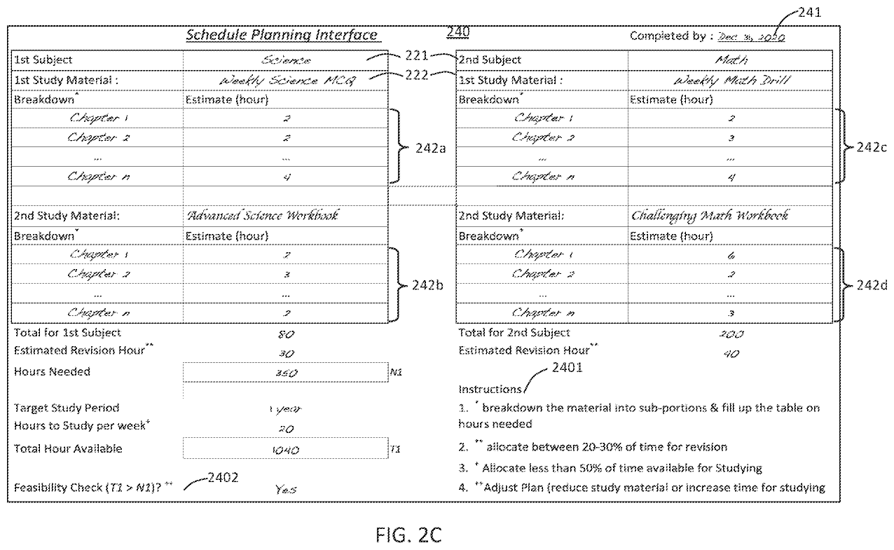

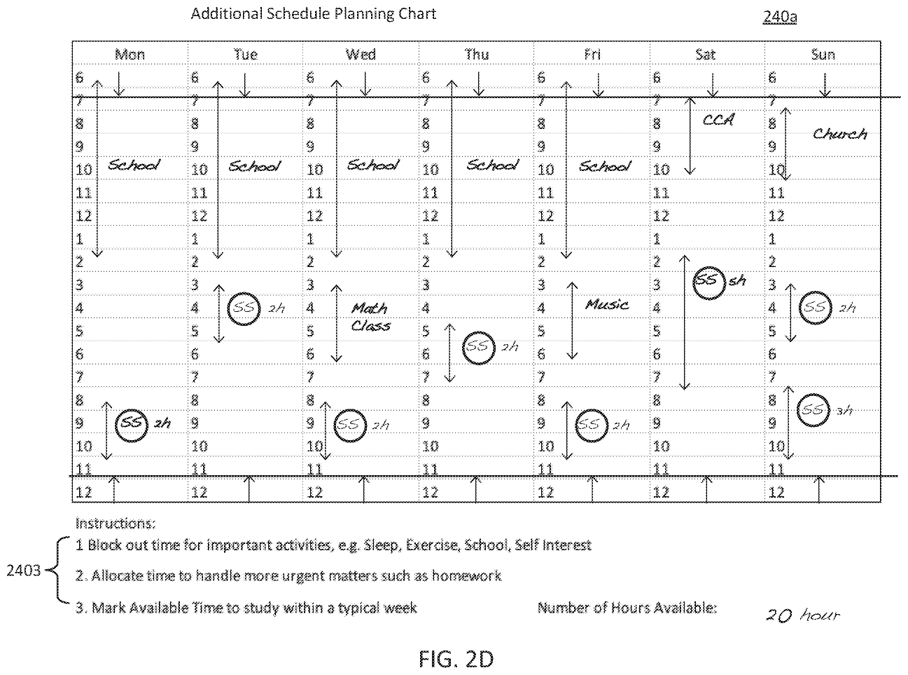

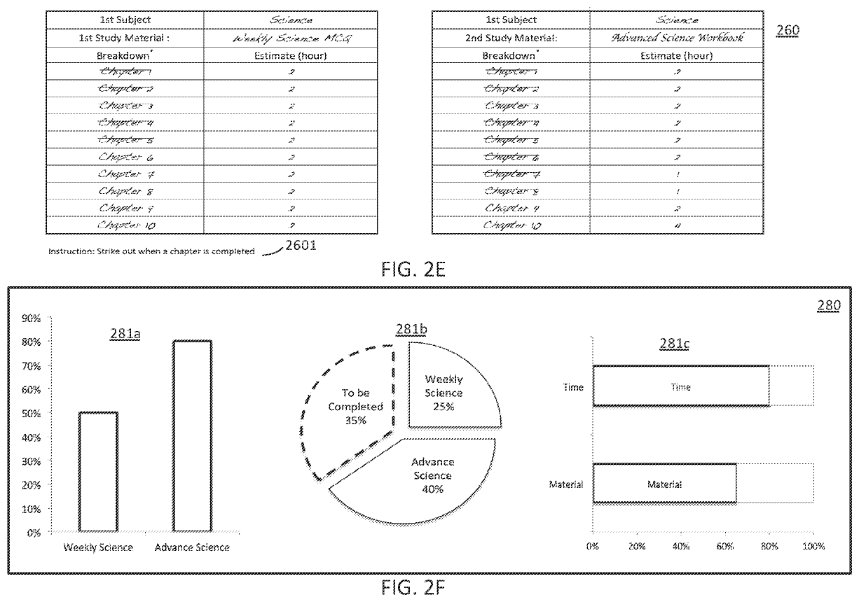

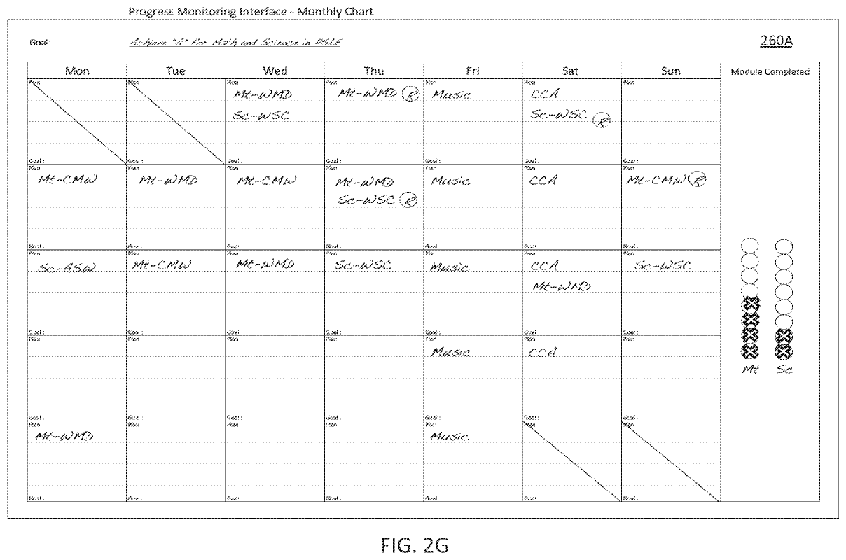

The study plan improvement proposal 171 may suggest to Mike to add his revision time, which forms the fourth schedule criterion 144, based on the revision status and the test scores of each chapter available respectively in the revision monitoring component 154 and the strength monitoring component 155 of the study plan 150. The study plan update interface 170 may be configured to increase revision time for topics that the student did relatively worse. The third planning criterion 123 may be incorporated into the study plan 150 by the target planning interface 120 for revision purposes when a predetermined condition is met, e.g. the student is found to be weak in some topics. For example, if the student is found to be weak in a topic of the at least one subject, contents from the similar topic in the third study material may be added into the study plan 150. In the above example, the LMS 100 may choose to add a third study material for the chapters that the students did badly. With the above, the LMS 100 may help students to learn more efficiently. The LMS 100 may be implemented in various ways, including but not limited to a paper-based planner system. The LMS 100 may also be implemented electronically through mobile devices such as smart phone and portable computing devices, as well as a terminal computing device. FIGS. 2A-2E illustrates various aspects of the interfaces 120, 130, 140 and 160 illustrated in FIG. 1. FIGS. 2A-2E are examples of such interfaces when implemented in a paper-based planner, however, FIGS. 2A-2E may be portions of screens of an electronically implemented system. FIG. 2A shows an illustrative example of the target planning interface 220, which comprises a first planning page having a planning table 2202.

FIG. 2A shows the inputs of a student in italic font. The planning table 2202 shown in FIG. 2A is very much simplified for illustration purpose. For illustration purpose, the scenario of Mike, explained in FIG. 1 is demonstrated in FIG. 2A. The schedule planning interface 240 may be further provided in a form of an electronic based interface where the student is provided with a list of study-material-candidates. The student is guided to select at least a portion of the plurality of study-material-candidates as the at least one study material. Either through the target planning interface 220 or an alternative study material selection interface, the LMS 100 may provide more information about the subjects and study material. FIG. 2B shows an example of an electronic based study material selection interface where prior use data is provided. All recommended materials are listed together with essential information grouped per the user-profile to fill up the planning table shown in FIG. 2A. In the example shown in FIG. 2B, the student may choose to select the use percentage group by top 10% of the student, which can be changed to either top, middle, or bottom 10%. Similarly, the cut-off of the 10% may be changed. The study material selection can be based on the background criteria of the students as shown in FIG. 2B. The criteria for background comparison can be customized. The fact that the target planning interface 220 prompted the student to input the information and study plan has several benefits. For example, most students ignore the planning process almost completely and just buy whatever study materials based on friend's recommendation. Some may buy too many and end up having insufficient time to complete the plan. By gathering and prompting the student to consider the hours needed upfront before studying will ensure feasibility check as well as the syllabus check. With the goal of the study plan clearly defined, the student will have more visibility as to the relevance of the selection of the study materials. For example, if a student aims for a distinction, he should select study materials used by other top students who did equally well. This cannot be done without a student interface as shown in FIG. 1 and FIG. 2B because without putting down the information together and compute a comparison, it is impossible to mentally carry out the process for the reason that different study materials group the contents differently. Even for one subject, the content is too much to be calculated without a tool such as the LMS 100.

The feasibility check of the plan initiated by the target planning interface 220 will go through the schedule planning. FIG. 2C shows an example of a schedule planning interface 240. The example as shown in FIG. 2C has been filled up by a student shown in italic font. The original interface with the table is shown in non-italic font. The schedule planning interface 240 comprises a scheduling table. The first target planning criterion 221, the second target planning criterion 222, and the second schedule criteria 242a-242d are arranged in rows or columns of the scheduling table. As illustrated in FIG. 2C, the first target planning criterion 221 shows the subject of the studies, which are Science and Mathematics. For each subject, the corresponding study materials Weekly Science MCQ, Advance Science Workbook, Weekly Math Drill, and Challenging Math Workbook are listed in the scheduling table. In order to better estimate the second scheduling criteria 242, the at least study material of the second planning criterion 222 may comprise a plurality of sub-portions. As shown in FIG. 2C, the sub-portions correspond to the chapters for each of the study materials. Dividing the sub-portions increases accuracy but the accuracy can be improved further through the study material selection interface that provides prior use data. The study material selection interface may form a part of the schedule planning interface 240 or the target planning interface 220.

The schedule planning interface 240 is configured to divide the study materials of the second planning criterion 242 into the plurality of sub-portions such that each of the plurality of sub-estimates 242a-242d is less than four hours, or between one and two hours for younger age group. This is to ensure that each of the plurality of sub-portions can be completed within a day. For students from younger age group, the schedule planning interface 240 is configured to divide the study materials such that each of the plurality of sub-estimates 242a-242d is between one and two hours. The schedule planning interface 240 is configured to divide the at least one study material into the plurality of sub-portions such that the plurality of sub-estimates 242a-242d differs less than 50% from each other. By making each of the plurality of sub-estimates 242a-242d substantially similar, an easier execution can be performed. In addition, making such arrangement illustrated above would increase accuracy of the time needed to complete the study materials. A feasibility check interface 2402 is provided to guide the student to compare a sum of the plurality of sub-estimates and the duration estimate so as to detect a conflict. FIG. 2D shows an additional planning chart 240a comprising a second portion of the schedule planning interface 240.

For a paper-based LMS, the table shown in FIG. 2A of the target planning interface 220 may be a combined progress monitoring and progress report generator interface 260. The paper-based LMS may comprise a first progress monitoring instruction configured to guide the student to perform a progress marking on the scheduling table upon completion of a portion of the at least one study material such that the scheduling table is adapted to show the first progress information. The student will provide input by striking a line on the portion of the study materials completed. The table then becomes the progress report which shows a progress information that is indicative of an amount of the study material completed by the student relative to the study plan. FIG. 2F shows an example of the progress report generator interface 280, which comprises three progress information 281a-281c. The first progress information 281a is indicative of the percentage of the work done for each of the study materials for the one subject. The second progress information 281b is indicative of the percentage of the work completed for each study material as compared to the incomplete portion. The third progress information 281c is indicative of a comparison between the percentage of the study materials completed and the percentage of time past or left behind. A longer bar for the graph for time is indicative of lagging behind as shown in FIG. 2F.

FIG. 2G shows an illustrative example of a progress monitoring interface 280 of a paper-based LMS. The progress monitoring interface comprises a short-term planner that listed a predetermined targeted short period. The predetermined targeted short period is one of a week, a fortnight and a month. Note that the subject and the study materials are written in an abbreviation form as discussed earlier. By using the abbreviation form, all the information will fit into the one-month planner in which the progress within the one month can be compared. In other embodiments where the short-term planner comprises a weekly or a bi-weekly planner, the study progress comparison will be made for one week or two weeks respectively. The arrangement of the information as shown in FIG. 2G which is suitable for a paper-based learning system may be beneficial in that the student may monitor his progress in various ways. First, as shown in FIG. 2G, the progress monitoring interface provides an interface for the student to compare progress for each subject he is taking. In the example shown in FIG. 2G, it is clear that the study on Mathematic (Mt) is significantly more than the subject of Science (Sc). The student may then decide whether this is intended. Second, the progress monitoring interface 280 may be configured to provide information about revision as the student marks down revision, shown in the letter ‘R’ circled. In this way, the progress monitoring interface 280 provides an interface for the student to compare an amount of time spent studying relative to an amount of revision. Third, the progress monitoring interface may provide the amount of study plan within the short-term planner. As shown in FIG. 2G, there are four cross marks for the subject of Mathematic and two cross marks for the subject of Science. In this way, the progress monitoring interface 280 provides an interface for the student to monitor amount of the study plan covered within the month, or the predetermined targeted short period of the short-term planner.

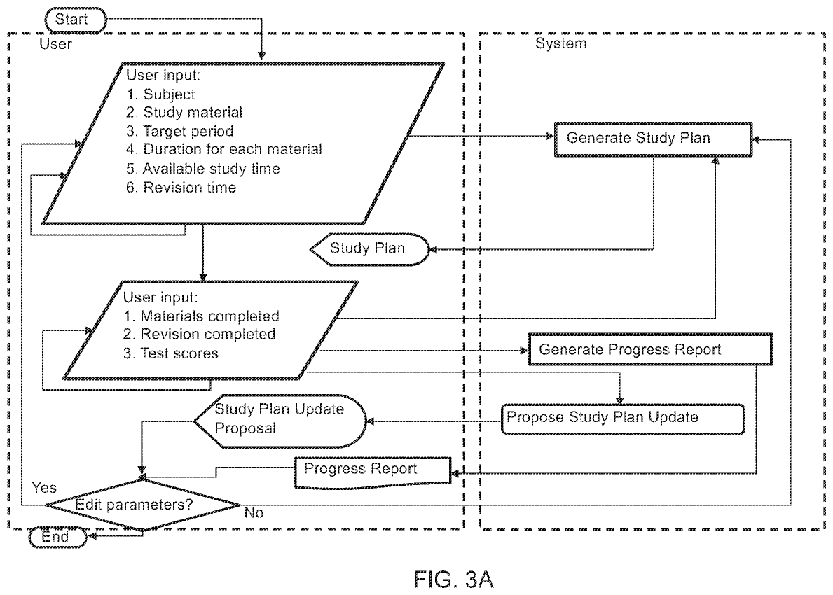

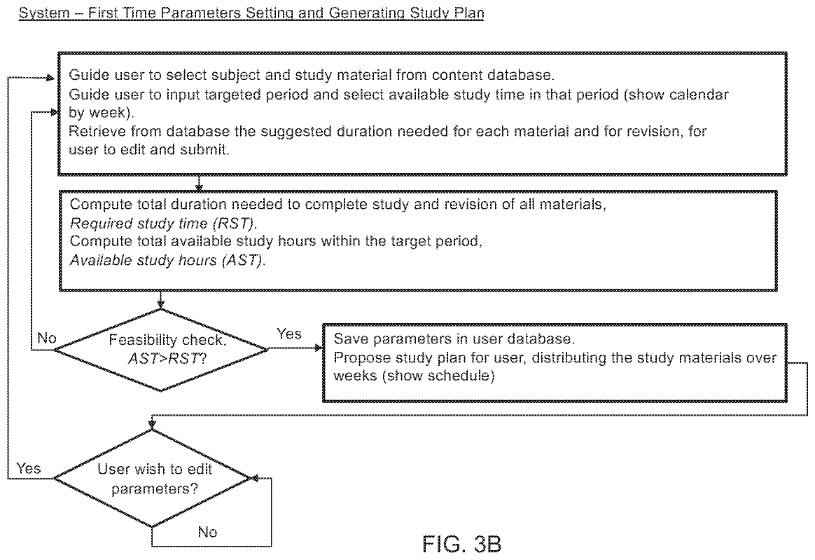

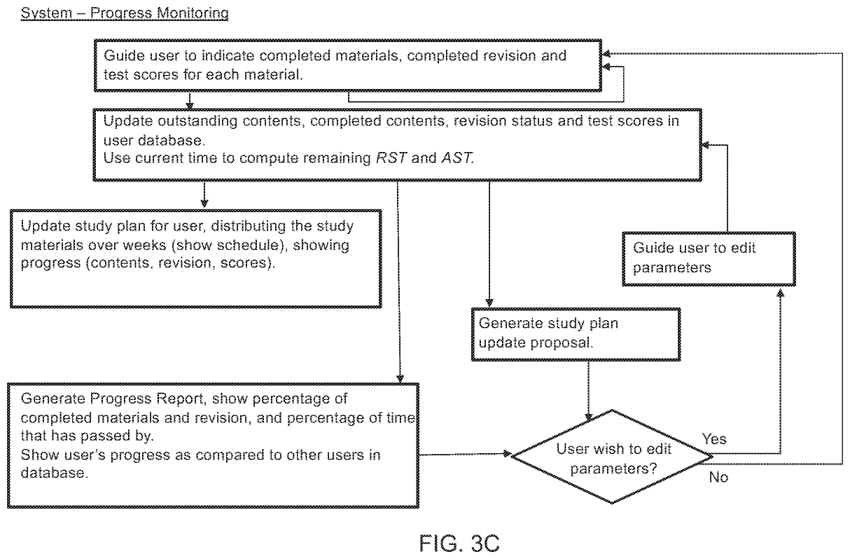

FIGS. 3A-3C shows a flow chart for implementing the method shown in FIG. 3. The method is for managing learning of a student. Managing learning includes at least one of the providing a study material, providing a study plan, tracking whether the student is able to complete the study plan on time, tracking the student's progress and results, and any other aspects or activities of the student related to learning. As shown in FIG. 3A, at the initial stage, the LMS receives student inputs related to the learning. The student inputs include one or more subjects that is targeted by the student, one or more study materials related to the one or more subjects, duration needed to study each of the one or more study materials, a targeted learning period and an available study time. The targeted learning period is from the start of a semester towards an exam, or a major exam, which ranges from three months to three years. The available study time comprises time period the student allocated for studying. Generally, the available study time is calculated by getting inputs from the student on how much time the student is willing to study within a week or a month, and thereafter compute the total available study time within the targeted learning period. The longer the targeted learning period, the more complex the plan would become. In general, most students of such LMS are for preparation of a major examination that requires at least 3 months of preparation. Therefore, a typical targeted learning period is more than three months. The LMS may provide guidance for determining the duration needed to study each of the study materials. For example, the LMS may provide information regarding choices of materials used, and time taken to complete the study materials sorted in accordance with prior-users in a similar grouping as described in previous embodiments. Prior users are students who used the LMS before the student used the LMS. In one embodiment, the prior-users comprises students who had sat for the examination in previous years. After gathering the inputs, optionally, the LMS may conduct a feasibility check by comparing the duration needed, and the available study time. Feasibility check at initial stage may be based solely on the prior use data and need improvement. Even if the feasibility check yield unfavorable outcome, the LMS may allow the plan to proceed until the student attempted the plan.

The LMS may generate a study plan that comprises a to-do list, and/or planner in a daily, weekly, bi-weekly, monthly and yearly plan. For computer base system, the study plan 150 comprises an estimation of study material and an estimation of time needed to complete the study material. The study plan 150 may comprise a plurality of time-estimates and respective groups of materials corresponding to the time-estimates. The study plan 150 may not need to be made explicit to the student. The student may provide further user-inputs related to a progress of the learning. The further user-inputs include portions of the one or more study materials completed by the student. The LMS will then prepare feasibility check following the flow chart shown in FIG. 3B and FIG. 3C. As shown in FIG. 3C, the LMS may categorize the study materials into a completed portion, and an outstanding portion. The outstanding portion may not be fixed by the LMS at any point of time and may be adaptively updated. One of the purposes of categorizing the study materials is to identify the outstanding content portion of the one or more study materials based on the further user-inputs, and subsequently computes an estimation of time needed to complete the outstanding content portion based on the duration needed to study each of the one or more study materials and the further user-inputs for feasibility check. In some embodiments, for feasibility check purposes, the LMS may compute remaining time, which corresponds to an amount of time remaining towards an end date of the targeted learning period. The feasibility check will then be completed by comparing the remaining time with the estimation of time needed to complete the outstanding content portion. As shown in FIG. 3B, the required study time (RST) will be the estimation of time needed. The available study time would be the remaining time available to study considering some time passed by since the plan is generated. The feasibility check may be performed adaptively by the LMS after a predetermined amount of progress information has been obtained. For example, for a yearly plan, the feasibility check will kick in only after a month. The further user-inputs comprise data identifying weak areas of the student. The LMS may add more time on weak areas to the estimated of time needed to complete the outstanding content portion. For example, the progress information may comprise test scores for the two or more subjects respectively. The LMS may add revision time for at least one of the one or more subjects when the test score for the at least one of the one or more subjects is below a specific threshold, or when the test score is unsatisfactory to the student.

FIG. 3A and FIG. 3C shows how the study plan can be updated. For example, the LMS may identify a completed content portion of the one or more study materials based on the further user-inputs and then compute an actual time used in order to study the completed content portion. Using the user-data, the LMS generates an improved estimation of time based on a comparison of the actual time used and the estimation of time needed. In this regard, the first time parameter setting may be revisited. The LMS may recommend a change to some parameters such as the available study time based on a comparison of the actual time used and the estimation of time needed. The LMS may also recommend a change to the one or more study materials based on a comparison of the actual time used and the estimation of time needed. If the parameters are accepted by the student, the LMS will then generate an improved study plan based on a comparison of the actual time used and the estimation of time needed.

Additional aspects of the present disclosure contemplate a method for managing learning of a student, comprising: (1) receiving user inputs related to the learning, the user inputs including one or more subjects that is targeted by the student, one or more study materials related to the one or more subjects, a targeted learning period and an available study time; (2) generating a study plan comprising an estimation for an identified study material of the one or more study materials, and an estimation of time needed to complete the identified study material; (3) receiving further user inputs related to a progress of the learning, the further user inputs including portions of the one or more study materials completed by the student; (4) identifying an outstanding content portion of the one or more study materials based on the further user inputs; and (5) computing an estimation of time needed to complete the outstanding content portion for feasibility check.

Additional aspects of the present disclosure contemplate the method further comprising computing a remaining time that corresponds to an amount of time remaining towards an end date of the targeted learning period.

Additional aspects of the present disclosure contemplate the method further comprising comparing the remaining time and the estimation of time needed to complete the outstanding content portion.

Additional aspects of the present disclosure contemplate the method further comprising adaptively performing a feasibility check based on the remaining time and the estimation of time needed to complete the outstanding content portion.

Additional aspects of the present disclosure contemplate the method further comprising receiving a test score for each of the one or more subjects respectively.

Additional aspects of the present disclosure contemplate the method further comprising adding revision time for at least one of the one or more subjects when the test score for the at least one of the one or more subjects is below a specific threshold.

Additional aspects of the present disclosure contemplate the method further comprising: (1) identifying a completed content portion of the one or more study materials based on the further user inputs; and (2) computing an actual time used in order to study the completed content portion.

Additional aspects of the present disclosure contemplate the method further comprising generating an improved estimation of time based on a comparison of the actual time used and the estimation of time needed.

Additional aspects of the present disclosure contemplate the method further comprising generating an improved study plan based on a comparison of the actual time used and the estimation of time needed.

Additional aspects of the present disclosure contemplate the method further comprising recommending a change to the available study time based on a comparison of the actual time used and the estimation of time needed.

Additional aspects of the present disclosure contemplate the method comprising recommending a change to the one or more study materials based on a comparison of the actual time used and the estimation of time needed.

Additional aspects of the present disclosure contemplate a learning management system comprising: (1) a target planning interface for receiving a first planning criterion comprising at least one subject targeted by a student, and a second planning criterion comprising at least one study material corresponding to the at least one subject; (2) a schedule planning interface for receiving a first schedule criterion comprising a targeted study period allocated for learning, and a second schedule criterion comprising a duration estimate for the student to complete the at least one study material; (3) a study plan generation interface for generating of a study plan based on the first planning criterion, the second planning criterion, the first schedule criterion and the second schedule criterion; (4) a progress monitoring interface for receiving a first progress input comprising a subset of the at least one study material completed by the student within a predetermined short period; and (5) a progress report generator interface for interactively generating a progress report providing a first progress information that is indicative of an amount of the at least one study material completed by the student relative to the study plan.

Additional aspects of the present disclosure contemplate the target planning interface is configured to receive a third planning criterion comprising an additional study material corresponding to the at least one subject, wherein study plan generation interface is configured to adapt the additional study material into the study plan when the student is ahead of the study plan by a predetermined margin.

Additional aspects of the present disclosure contemplate the study plan generation interface is configured to adapt a sub-topic of the additional study material into the study plan for revision purposes if the student did badly in the sub-topic.

Additional aspects of the present disclosure contemplate the schedule planning interface is adapted to receive a third schedule criterion comprising an estimate of time available for learning within the predetermined short period.

Additional aspects of the present disclosure contemplate the schedule planning interface comprises a feasibility check interface providing an indication of feasibility of the study plan.

Additional aspects of the present disclosure contemplate the target planning interface is configured to provide prior use data for guiding the student to select the at least one study material from a plurality of study materials.

Additional aspects of the present disclosure contemplate the target planning interface is configured to provide a piece of statistical information based on the prior use data by other students for at least one of the plurality of study materials.

Additional aspects of the present disclosure contemplate the study plan comprises a revision monitoring component that is indicative of an amount of time spent on doing revision.

Additional aspects of the present disclosure contemplate the progress report generator interface is adapted to generate a third progress information that is an indicative of a comparison between a percentage of the amount of the at least one study material completed by the student, and a percentage of time that has passed by as compared to a remaining time towards the targeted study period.

Additional aspects of the present disclosure contemplate the progress report generator interface is adapted to generate a fourth progress information that is an indicative of a comparison of the first progress information of the student and a corresponding first progress information of a different student or a group of students.

Additional aspects of the present disclosure contemplate the schedule planning interface is configured to divide the at least one study material of the second planning criterion into a plurality of sub-portions that can be completed by the student within 4 hours.

Additional aspects of the present disclosure contemplate the schedule planning interface is configured to divide the at least one study material of the second planning criterion into a plurality of sub-portions such that each of the plurality of sub-portions differs less than 50% from each other.

Additional aspects of the present disclosure contemplate the progress monitoring interface comprises a short-term planner that lists a predetermined targeted short period, wherein the predetermined targeted short period is one of a week, a fortnight and a month.

Additional aspects of the present disclosure contemplate the progress monitoring interface comprises a first monitoring interface for the student to compare progress between the at least one subject and one additional subject.

Additional aspects of the present disclosure contemplate the LMS periodically checks whether the student is able to complete the study plan within the targeted study period.

### Computer System Based LMS

FIG. 4 shows a block diagram of instruction sets of an LMS 400. The LMS 400 comprises at least one processor and a memory coupled to the at least one processor. The at least one processor may be configured to execute instruction sets illustrated in the illustration set block diagram shown in FIG. 4. The LMS 400 provides a complete learning management to a student, including but not limited to, getting suitable study materials, tutorial, or even tutors for a student. A student refers to a human subject who uses the LMS 400 to achieve a learning outcome or in a simple language, to learn something as outlined in a syllabus of the LMS 400. For most cases, the student will eventually sit for an examination. The syllabus of the LMS 400 may optionally made explicit, but generally a syllabus of the LMS 400 follows requirement of examinations that the student will sit for. Similar to the word student, the word “tutor” refers to anyone who coaches or teaches something to the student (not limited to schoolteacher or anyone taking a teaching role). All materials are available online and the tutor would include anyone who has an interest in the learning process of the student, who could be conducting activities such as generating class tutorial, marking the assessment questions, mentoring, coaching, suggesting teaching materials, motivating the student to achieve better results and any other roles that benefit the students. The tutor may also include the parents of the students. For example, the LMS 400 may manage scheduling similar to the embodiment shown in FIG. 1. The LMS 400 may also provide various prior use data for assisting teachers or tutors to teach in a more efficient way. In addition, the LMS 400 may assist content suppliers to provide better study materials and the LMS 400 may also have machine-learning capabilities to provide customized study materials or customized learning plan for the student. Traditionally, the content-suppliers include publishers, or teachers who produce assessment questions. For LMS 400, the content-supplier may include anyone who may produce a study material for the students. The content-supplier may also include students who tweak some questions for other students to study, and teachers who may be inspired to set some assessment questions or material for students to learn. As shown in FIG. 4, the LMS 400 comprises a database 410, user-interface component 420, a user-management component 430, a provider management component 440, a rating administration component 450, a main controller 460, and a prior use information processing component 470. The instruction sets for a specific functionality is grouped together as shown as in various components 410, 420, 430, 440, 450, 460, and 470 which were grouped per functionality. One or more of the various instruction sets or components 410-470 may be stored as instructions in computer memory and may be executable by a processor of the LMS 400. In some embodiments, the memory of the LMS 400 may include a non-transitory computer-readable medium having program code recorded thereon for controlling access to a device or a plurality of devices. The program code may be executed by one or more processors of the LMS 400 to perform various functionalities of instruction sets or the components 410-470. In some embodiments, the LMS 400 may alternatively or additionally include a main server and a plurality of user-devices, each has one processor and a memory. The main server may perform the functionality of the main controller 460, rating administration component 450, provider management component 440 and storing the database. The plurality of user-devices may perform the functionality of user-interface component 420 and a portion of the user-management component 430.

The database 410 are records stored in the memory of the LMS 400. The database 410 may comprise content database 412, user-database 414, rating database 416 and prior use database 418. The content database 412 comprises a plurality of study materials for selection by the student. The user-database 414 comprises all relevant data from all users. The rating database 416 comprises ratings assigned for each student as well as ratings for each of the plurality of study materials. The ratings may be used to assist the LMS 400 to produce a customized study material for the students. The prior use database 418 comprises usage statistics or usage data of the plurality of study materials by all students. Through the prior use information processing component 470, the prior use database is analyzed so as to compile or deduce information needed to improve both teaching and learning. The rating administration component 450 updates and analyze the rating database 416.

As explained above, the LMS 400 may be an integrated system used by students, teachers as well as content providers. The students, teachers and the content providers access the LMS 400 through user-interface component 420. The user-interface component 420 comprises instruction sets executable by the processor so as to interface with a student, a tutor and/or a content supplier. The user-interface component 420 may be a single computer system or may be a distributed system having multiple computer systems and/or handheld mobile devices used by anyone to access the LMS 400. The user-interface component 420 comprises a student interface component 422, a tutor interface component 424 and a supplier interface component 428. Through the student interface component 422, the LMS 400 receives inputs from a student and display, to the student, various information from the LMS 400. In one embodiment, the student interface component 422 includes instructions to provide interface for the student to create and manage study plan, to take on assessment questions, to load a scanned copy of the assessment questions attempted, to receive or provide progress reports and other activities as a role of a student. Through the tutor interface component 424, the LMS 400 receives inputs from a tutor and to display, to the tutor, various information from the LMS 400. The tutors are usually teachers whose job is to provide guidance for the student. The tutor interface component 424 comprises instruction sets to provide interfaces to facilitate teaching, as well as for the tutor to view results or other information of the students. The tutor interface component 424 also includes instruction sets to provide interfaces needed for the tutor to provide coaching and for communicating with a student either via text or video interface. The tutor's role may be more of a coach who oversees the progress of the student and provides guidance and counseling. The tutor may (but not limited to) conduct tutorial lessons because in a system such as the LMS 400, tutorial classes may comprise video lessons which are referred to as a teaching material. The tutor may get access to the LMS 400 and through the tutor interface component 424, the tutor may obtain various data such as prior use data so as to give advices to the students. Through the content supplier interface component 428, the LMS 400 receives inputs from the content suppliers, and display, to the content suppliers, various information from the LMS 400. The content supplier may obtain various statistics from prior use data so that the content supplier knows where to improve on the study material, or to create a more relevant study material. The content supplier, traditionally, are producers of assessment books or publishers of such teaching material. For LMS 400, the content suppliers may also comprise teachers who produce lesson tutorials to the students.

Some users of the LMS 400 may have multiple roles. For example, a student may also produce tutorial service to other users and may also product teaching material, which is the role of a content supplier. The student can also be a coach for other students, in which the role would be a tutor under the context of LMS 400. Similarly, a tutor may also use the LMS 400 to learn some other subject and may take on the role of a student as well as the content supplier. The content supplier may also have other roles such as a student and a tutor depending on how the content supplier intends to use the LMS 400. However, depending on which role a user plays, the user will take on various interfaces 422, 424 and 428 for various essential tasks. The interfaces 422, 424 and 428 may be provided through the user-interface components 420. In one embodiment, a portion of the user-interface component 420 may be executable using a device owned by the user such as a personal computer, a smart phone, a mobile computing device or other similar devices, while other instruction sets or component are executable by a remote server that host the LMS 400.

The user-management component 430 may comprise a plurality of sub-components related to student's activities. For example, the user-management component 430 may comprise a target planning component 432, a scheduling component 434, a study plan administration component 435, and a progress monitoring component 436. The target planning component 432, the scheduling component 434, the study plan administration component 435, and the progress monitoring component 436 are instruction sets for creating and managing the study plan. In addition, the user-management component 430 may also comprise a content selection component 437, a welfare management component 439, an assessment test management component 438 and a result forecast component 431, which are components for managing learning contents such as study material selection or assessment test related matters.

The provider management component 440 comprises a content administration component 442, a syllabus administration component 444, and a payment management component 446. The content administration component 442 facilitates management of the content database 412. For example, the content administration component 442 receives, from the content-supplier interface component 428, a plurality of content proposals so as to be stored in the content database 412 for selection by the students. The content proposals comprise a plurality of study materials such as assessment questions, tutorials, videos and any other material used for learning the subject. The content administration component 442 may accept or reject the content proposals depending on criteria predetermined in the LMS 400. The content administration component 442 may also ensure that the plurality of study material proposals are provided in a specific format as outlined in a syllabus administrated within the syllabus administration component 444. The syllabus administration component 444 comprises interfaces for the tutors or educators to provide inputs to the syllabus of learning subjects. Each subject may comprise a plurality of topics needed as outlined in a syllabus. A syllabus is usually outlined for any examinations to describe the scope and the learning outcome. A syllabus usually includes a plurality of topics, which is arranged in chapters or sub-chapters in many study materials. The student will be examined on the plurality of topics. For avoidance of doubt, the plurality of topics referred in this specification may not include everything an examination board specified, but selected in a way deemed suitable by educators. The payment management unit enables a payment process where the tutors and the content suppliers are paid when the teaching contents or teaching services are used. In this way, tuitions by tutors, as well as teaching materials by various suppliers, can be used within a single platform.

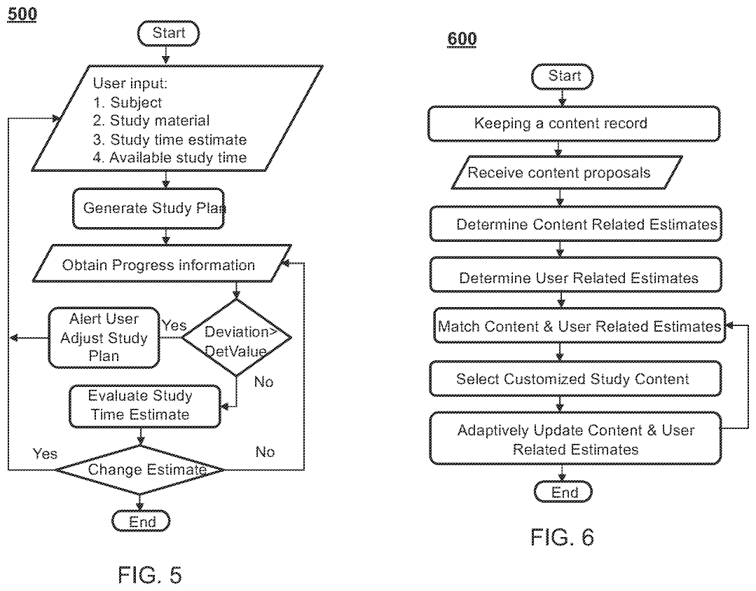

The main controller component 460 may execute instruction sets for coordinating other instruction sets described in the components 410-470. The LMS 400 may be configured to provide several functionalities depending on the instruction sets or the program desired by the student. For example, the LMS 400 may be utilized to perform a method for efficient schedule planning and monitoring illustrated in FIG. 5. Unlike conventional methods where most students do not even have a plan, the method utilizing LMS 400 includes machine learning and data analyzing to ensure an efficient study plan as well as the timely execution of the study plan.

### I Schedule Planning and Monitoring

FIG. 5 shows a method 500 for managing learning plan of a student who is studying at least one subject for a targeted study period through the LMS 400. The targeted study period may be the time period between a start date and an end date of the studying. The method 500 comprises receiving, from a student, user-inputs such as at least one study material corresponding to the at least one subject, a study time-estimate that corresponds to a duration needed to complete the at least one study material, and an available time-estimate that corresponds to time available for studying within the targeted study period. The at least study materials comprises materials that the student will use to study for the at least one subject which may include lessons, tutorial, assessment questions that are provided through books or online portals or the LMS 400. The study time-estimate includes an estimation as to how much time is needed to complete the at least one study material. The available time-estimate includes the estimated time that the student will study during the targeted study period. The available time-estimate may be optionally computed by the LMS 400 by seeking from the student, on a weekly or a monthly basis, the time period the student is willing to study as shown in FIG. 2D. For the LMS 400, the study time-estimate may be provided by LMS 400. Using the example of Mike, the inputs may be the subjects of Science and Math, the study materials such as Weekly Science MCQ and Advanced Science Workbook for Science; and Weekly Math Drill and Challenging Math Workbook for Mathematics, as well as the study time estimates and the available study time-estimate shown in FIG. 2C. Unlike conventional way to select a study material, the student will be guided to select the at least one study material from the content database 412 that stored a plurality of study-material-candidates together with the prior use data. For a computer system based LMS 400, the at least one study material may not be necessary chosen from assessment books but may comprise electronic based assessment questions or other study materials from a plurality of suppliers. The selection of the at least one study material will be described in subsequent embodiments. The prior use data of the plurality of study-material-candidates comprises usage statistics collected by the LMS 400 from other students who had used the LMS 400. For example, the usage statistics comprises a histogram as to how many students use each of the plurality of study-material-candidates. The prior use data may be computed and processed by the LMS 400 so as to guide the student to select the at least one study material from the plurality of study-material-candidates. For example, a student may make an enquiry to the LMS 400 to display the usage statistics of a specific group of students who has similar strength or background. In this way, the student will be guided to select a study material that is more suitable to his strength instead of a predetermined sets chosen in an assessment book. The prior use data may also comprise the study time-estimate for each of the plurality of study-material-candidates. In this way, the method shown in FIG. 5 will be beneficial to the student because the student will get some insights as to whether the study-material-candidate that he selects is suitable, and whether the student is able to complete the study-material-candidate within the limited time if he chooses the study-material-candidate. The at least one study material decided by the student may be provided in a printed format, or in an electronic format printable by the student.

Next, the method 500 proceeds to generate a study plan. The study plan 150 comprises an estimation of study material and an estimation of time needed to complete the study material. For example, the study plan may comprise a proposed to-do-list with an estimated time and date to complete a specific selected amount of study materials that may comprise a set of assessment question. The specific selected amount of study materials is selected such that the student is able to complete within a typical time window of 1-2 hours. For example, the study plan may include a set of assessment questions identified for the students with time-estimates. The LMS may then select a fixed amount of the assessment questions from the entire set. For a student who plans to learn more than one subject, the at least one study material for each subject may be divided into a plurality of sub-chapters, or even a smaller portion that can be completed within 1-2 hour. For students at younger age, each of the plurality of sub-chapters will be planned to be completed within 30 minutes or less. In other words, the LMS 400 will be used to determine a sub-duration of time estimate for each of the plurality of sub-chapters prior to the study plan generation. In the embodiment shown in FIG. 5, the sub-duration of time estimates for each of the plurality of sub-chapters are substantially similar and are usually age dependent. The at least one study material has to be broken down accordingly such that the sub-duration of time estimate are substantially similar.

In other embodiment, the sub-duration of time-estimates may be different but differs from each other for not more than 400% for ease of planning. For example, the study materials may be broken down to a smaller quantity that can be completed with an hour for one subject, but for another more complicated subject, the study materials may be broken down to a larger quantity that can be completed within four hours. The study plan may comprise a weekly, a biweekly, or a monthly planner having a to-do-list with each of the plurality of sub-chapters being listed down in the planner for the student to study accordingly. With prior use data, the accuracy of the sub-duration of time estimate can be improved. For study material with video lessons, the time estimates may be extracted from the length of the video lesson through the LMS 400. For example, instead of a generic 1-2-hour estimate, the LMS 400 may be able to provide data as to the actual time spent by other students who are stronger or weaker. For example, the LMS 400 may provide information to the student such as the actual time used by a top percentage or a bottom percentage of prior students to the student. In this way, the student will have a better accurate estimate as the student would know how fast he does as compared to top or bottom percentage of students.

Next, the method 500 proceeds to receive a first progress information when the student finishes studying a portion of the at least one study material. The first progress information comprises information related to what the student had accomplish, by when the student accomplishes, and how much time needed the student used to accomplish a portion of the study plan. The first progress information may be automatically received when the student studies the at least one study material through the LMS 400, or when the student gives an input through the student interface component 422. The LMS 400 may adaptively compute feasibility check of the study plan to ensure that the student is able to complete the study material, or to cover a desired portion of the syllabus on time to prepare the examination. For example, the LMS 400 may compute the feasibility check by triggering the check every time a progress information is received, and/or at a periodically basis such as on a daily or weekly basis. As shown in FIG. 5, the LMS 400 adaptively check whether there is a deviation between the study plan and the first progress information. If the deviation is larger than a predetermined threshold value (netValue), the LMS 400 will alert the student to adjust the study plan. For example, for the month of March, Mike is supposed to complete three topics for the subject of Science per his study plan, but Mike only completed one topic within the first three month. There is a deviation of two topics. The LMS 400 may compute the deviation in terms of time-estimates, and not in the form of number of topics, in order to achieve accuracy and consistency. For example, the LMS 400 may compute the time-estimates needed to complete the three topics and quantify the ‘behind schedule of two topics’ in a specific number of hours of the study plan. If the predetermined value for the deviation is more than the specific deviation threshold the LMS 400 will alert Mike to revisit the study plan, either to increase his study time or to reduce some study materials that he plans to complete initially. In the embodiment shown in FIG. 5, the deviation threshold is less than five hours. In general, a student is able to study on his own 10-20 hours a week and being behind schedule for more than five hours may require 2-3 weeks to recover the time lost. The ability of LMS 400 to detect a deviation up to the precision of hours is beneficial because the student will be reminded by LMS 400 with an accurate quantity amount of time, which is something not available using conventional study method without the use of the LMS 400 that comprises time-estimates.

In addition to the feasibility check, the method 500 includes a step to evaluate the study-time estimate so as to improve the accuracy for the study time-estimate. For example, whenever a progress information is received, the LMS 400 automatically computes the actual time used by the student and the planned time as stated in the study plan. If the actual time used is consistently faster or slower than the planned time, the study time-estimate can be updated. In this way, the LMS 400 improves the study time-estimate in accordance with an actual time used for completing a sub-portions of the at least one study material by the student.

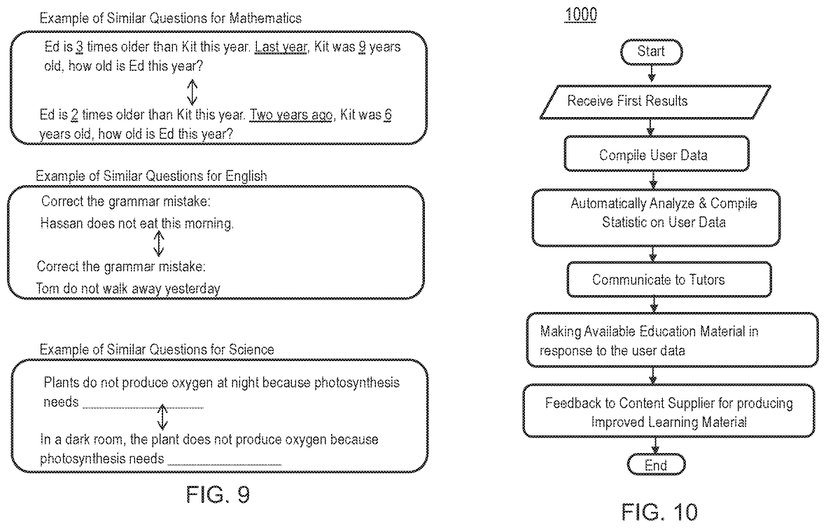

In one embodiment, the study time-estimate includes a learning time component and a revision time component. The learning time is the time duration needed to go through the at least one study material whereas the revision time component is the time duration allocated for doing revision. Doing revision is a key activity in learning because revision strengthens understanding of the student on the subject matter of his learning. The revision may include deliberate practice compilation as shown in FIG. 8 and FIG. 9. If a student understands the topic well, less revision will be needed. In other words, a student with an above average examination results or who knows the subject matter better (referred hereinafter as “stronger student”) would require less revision relatively as compared to an average student. On the other hand, another student who has a lower than average examination results (referred hereinafter as “weaker student”) usually has less knowledge of the subject matter and would require more revision as compared to an average student. When the LMS 400 receives an assessment result of the student related to the at least one subject, the LMS 400 may automatically compute the revision time needed and regenerate the study plan by adjusting the revision time component of the study time-estimate as necessary. The more progress information received, a better estimate may be produced by the LMS 400. Having a better study time-estimate by the LMS 400 will ensure that the student utilizes his time on studying more efficiently. The LMS 400 adaptively adjust the study time-estimate either (i) as-and-when a progress information is received or (ii) at a specific time intervals depending on which way is shorter. In this way, the LMS 400 will assist the student to complete the study plan before an end date of the targeted study period. The end date of the targeted study period is usually a date before a major examination. If the student does better than what he predicted, the visibility of having more time to accomplish more would enable the student to add more study materials to his plan.

There are a few ways to conduct the feasibility check. One way to conduct a feasibility check is to first compute the total outstanding available time and the total outstanding study time-estimate for all the outstanding study materials not yet completed by the student, and then check that the total outstanding study-time estimate is shorter than the total outstanding available time. Both the study-time estimate, and the total available time can be updated from time to time. A second way to conduct feasibility check is by comparing completed portion and outstanding portion. For example, the LMS 400 may first categorize the at least one study material into a completed content for portions of the at least one study material completed by the student, and an outstanding content for portions of the at least one study material not yet studied by the student. Next the LMS 400 computes (1) a remaining content percentage that indicates a percentage of the outstanding content relative to a sum of the completed content and the outstanding content; and (2) a remaining time percentage that indicates a ratio of an amount of time remaining towards an end date of the targeted study period relative to the targeted study period. The LMS 400 adaptively checks whether the student is able to complete the study plan within the targeted study period by comparing the remaining content percentage and the remaining time percentage. A behind-schedule warning may be triggered when the remaining content percentage is more than the remaining time percentage by a first predetermined margin. Similarly, the LMS 400 may flag an ahead-schedule alert when the remaining time percentage is more than the remaining content percentage by a second predetermined margin.

A small deviation is expected but when the deviation is significant, for example, by two weeks, the LMS 400 may propose, to the student, to reduce the at least one study material or to increase the available time-estimate when the student is significantly behind schedule. On the contrary, the LMS 400 may propose, to the student, to add an additional study material to the at least one study material when the student is significantly ahead of the planned schedule. One way to motivate the student to work harder or to take more time, is by showing progress comparison. For example, the LMS 400 may display, to the student, a progress comparison showing the remaining content percentage as compared to the remaining time percentage. The progress comparison will give an indication to the student whether he is ahead of schedule or behind schedule. Some students may not take action to study although they are behind schedule. For such students, the LMS 400, may optionally display, to the student, a statistic of the progress comparison of the student as compared to a plurality of all other students who are sitting for the same examination, or a smaller group of other students within criteria agreed by the student. The group comparison will motivate the student.

Most students will take two or more subjects in an examination. In some extreme cases, the students may be taking more than ten subjects and ensuring timely execution of the study plan may be not as straightforward. To make the matter worse, each subject usually requires more than one study materials. For most cases where the student is taking two or more subjects, where the student needs to complete two or more study materials corresponding to the two or more subjects, the LMS 400 categorize the two or more study materials for each of the two or more subjects into a completed content for portions of the at least one study material completed, and an outstanding content for portions of the at least one study material not studied respectively for each of the two or more subjects. Next, the LMS 400 computes a subject remaining content percentage that corresponds to a percentage of the outstanding content for each of the two or more subjects. A comparison of the subject remaining content percentage between the each of the two or more subjects may be then presented to the student. In this way, the student may be able to manage his studies so as not to overly emphasis on the favorite subjects, and it ensures that the student is able to cover the study plan for all subjects.

The process of receiving progress information, feasibility of the study plan check and the evaluation of the study time-estimate, generation or regeneration of the study plan may continue until the student completes the entire study plan or when an end date of the targeted study period arrives. As known by a person skilled in the art, the method 500 may be implemented using a computer system that has at least one processor and a memory coupled to the at least one processor. The processor may then execute instruction sets illustrated in the student interface component 422, the study plan admin component 435, the target planning component 432, the scheduling component 434, the progress monitoring component 436 and other components in the user-management component 430 shown in FIG. 4.

Additional aspects of the present disclosure contemplate a method for managing learning plan of a student who is studying at least one subject for a targeted study period, the method comprising: (1) providing an LMS for the student to provide user inputs, wherein the user inputs comprise: (a) at least one study material corresponding to the at least one subject; (b) a study time-estimate that corresponds to a duration needed to complete the at least one study material; and (c) an available time-estimate that corresponds to time available for studying within the targeted study period; (2) generating, with the LMS, a study plan; (3) receiving, at the LMS, a first progress information when the student finishes studying a portion of the at least one study material; (4) identifying, with the LMS, whether there is a deviation between the study plan and the first progress information; and (5) alerting the student to adjust the study plan when the deviation is larger than a predetermined deviation threshold value.

Additional aspects of the present disclosure contemplate the method further comprising adaptively computing, with the LMS, a feasibility check on the study plan as to whether the at least one study material can be completed before an end date of the targeted study period.

Additional aspects of the present disclosure contemplate the LMS periodically checks whether the student is able to complete the study plan within the targeted study period.

Additional aspects of the present disclosure contemplate the method further comprising: (1) displaying, to the student, a plurality of study-material-candidates and prior use data of the plurality of study-material-candidates; and (2) guiding the student to select the at least one study material from the plurality of study-material-candidates.

Additional aspects of the present disclosure contemplate the prior use data of the plurality of study-material-candidates comprises usage statistics collected by the LMS from other students, wherein the usage statistics comprises information to guide the student to select the at least one study material from the plurality of study-material-candidates.

Additional aspects of the present disclosure contemplate the study time-estimate is computed by the LMS based on the prior use data.

Additional aspects of the present disclosure contemplate the method further comprising recalculating, with the LMS, the study time-estimate in accordance with an actual time used for completing a sub-portion of the at least one study material by the student.

Additional aspects of the present disclosure contemplate the study time-estimate comprises a learning time component that corresponds to a first amount of time needed to go through the at least one study material, and a revision time component that corresponds to a second amount of time needed to do revision.

Additional aspects of the present disclosure contemplate the method further comprising receiving an assessment result related to the at least one subject and regenerating the study plan by increasing the revision time component of the study time-estimate when the assessment result is unsatisfactory.

Additional aspects of the present disclosure contemplate the method further comprising dividing the at least one study material into a plurality of sub-chapters and determining a sub-duration of time-estimate for each of the plurality of sub-chapters.

Additional aspects of the present disclosure contemplate the study plan comprises a weekly, biweekly or a monthly planner.

Additional aspects of the present disclosure contemplate the method further comprising categorizing the at least one study material into a completed content for portions of the at least one study material completed by the student, and an outstanding content for portions of the at least one study material not yet studied by the student.

Additional aspects of the present disclosure contemplate the method further comprising computing a remaining content percentage that indicates a percentage of the outstanding content relative to a sum of the completed content and the outstanding content.

Additional aspects of the present disclosure contemplate the method further comprising computing a remaining time percentage that indicates a ratio of an amount of time remaining towards an end date of the targeted study period relative to the targeted study period.

Additional aspects of the present disclosure contemplate the method further comprising providing, through the LMS, a progress comparison showing the remaining content percentage as compared to the remaining time percentage.

Additional aspects of the present disclosure contemplate the method further comprising communicating, to the student, a statistic of the progress comparison of the student as compared to the progress comparison of a plurality of additional students.

Additional aspects of the present disclosure contemplate the LMS adaptively checks whether the student is able to complete the study plan within the targeted study period by comparing the remaining content percentage and the remaining time percentage.

Additional aspects of the present disclosure contemplate the method further comprising flagging a behind-schedule warning when the remaining content percentage is more than the remaining time percentage by a first predetermined margin.

Additional aspects of the present disclosure contemplate the further comprising recommending, to the student, to reduce the at least one study material or to increase the available time-estimate.

Additional aspects of the present disclosure contemplate the method further comprising flagging an ahead-schedule alert when the remaining time percentage is more than the remaining content percentage by a second predetermined margin.

Additional aspects of the present disclosure contemplate the method further comprising proposing, to the student, to add an additional study material to the at least one study material.

Additional aspects of the present disclosure contemplate the at least one subject comprises two or more subjects, and the at least one study material comprises two or more study materials corresponding to the two or more subjects.

Additional aspects of the present disclosure contemplate the method further comprising causing the LMS to categorize the at least one study material for each of the two or more subjects into a completed content for portions of the at least one study material completed by the student, and an outstanding content for portions of the at least one study material not studied by the student respectively for each of the two or more subjects.

Additional aspects of the present disclosure contemplate the method further comprising: (1) computing a subject remaining content percentage that corresponds to a percentage of the outstanding content for each of the two or more subjects; and (2) communicating to the student, through the LMS, a comparison of the subject remaining content percentage between the each of the two or more subjects.

Additional aspects of the present disclosure contemplate the method further comprising: (1) receiving an assessment result for each of the two or more subjects; and (2) providing an additional revision recommendation to the student for revising the study plan based on the assessment result.

Additional aspects of the present disclosure contemplate the method further comprising providing a comparison of time spent on studying and time spent on doing revision for each of the at least one subject.

Additional aspects of the present disclosure contemplate the method further comprising adaptively adding or reducing the at least one study material in accordance with a projection computed based on the first progress information.

Additional aspects of the present disclosure contemplate a computer system for providing an LMS to a student who is studying at least one subject for a targeted study period, the computer system comprising a memory and at least one processor coupled to the memory, the at least one processor is configured to: (1) store a record of user inputs, wherein the record of user inputs comprise: (a) at least one study material corresponding to the at least one subject; (b) a study time-estimate that corresponds to a duration needed to complete the at least one study material; and (c) an available time-estimate that corresponds to time available for studying within the targeted study period; (2) generate a study plan, wherein the study plan comprises a time schedule of the student to complete the at least one study material; (3) receive a first progress information when the student finishes studying a portion of the at least one study material; (4) identify whether there is a deviation between the study plan and the first progress information; and (5) provide an alert to the student so that the student consider adjusting the study plan when the deviation is larger than a predetermined deviation threshold value.

Additional aspects of the present disclosure contemplate the at least one processor is further configured to adaptively perform a feasibility check on the study plan as to whether the at least one study material can be completed before an end date of the targeted study period.

Additional aspects of the present disclosure contemplate the at least one processor is further configured to categorize the at least one study material into a completed content for portions of the at least one study material completed by the student, and an outstanding content for portions of the at least one study material not yet studied by the student.

Additional aspects of the present disclosure contemplate the at least one processor is further configured to: (1) compute a remaining content percentage that indicates a percentage of the outstanding content relative to a sum of the completed content and the outstanding content; and (2) compute a remaining time percentage that indicates a ratio of an amount of time remaining towards an end date of the targeted study period relative to the targeted study period.

Additional aspects of the present disclosure contemplate the at least one processor is further configured to flag a behind-schedule warning when the remaining content percentage is more than the remaining time percentage by a predetermined warning threshold.

Additional aspects of the present disclosure contemplate the at least one processor is further configured to reduce the at least one study material or to increase the available time-estimate when the remaining content percentage is more than the remaining time percentage by a predetermined behind-schedule threshold.

Additional aspects of the present disclosure contemplate the at least one processor is further configured to flag an ahead-schedule alert when the remaining time percentage is more than the remaining content percentage by a predetermined alert margin.

Additional aspects of the present disclosure contemplate the at least one processor is further configured to add an additional study material to the at least one study material when the remaining time percentage is more than the remaining content percentage by a predetermined ahead-schedule margin.

### II Study Material Selection and Compilation

In addition to the schedule planning and monitoring, the LMS 400 can be configured to improve the study material selection and compilation according the method 600 shown in FIG. 6. FIG. 6 shows a method 600 for providing a customized study content. Conventional way of getting study materials is by having a generic assessment book or a generic program stored in a software for learning. In contrast, the method 600 shown in FIG. 6 may utilize machine learning to adaptively compile or select suitable materials for each individual student. The machine learning may be accomplished through analyzing prior use data, and prior usage trend to deliver a more suitable study material as will be discussed subsequently. As shown in FIG. 6, the method 600 starts with receiving at least a content proposal from one or more content suppliers for the students to select as the study material. The one or more content suppliers receive from the LMS 400, or from an external sources, a syllabus. The syllabus is the scope of a major examination or a scope of the learning program where the students are targeting. The syllabus may comprise a plurality of topics. For some examinations, the syllabus or the scope of the examination may be provided by the examination board. However, the plurality of topics in the LMS 400 may not follow entirely the way the examination board specify, but in a way deemed fit but educators or tutors. The syllabus, as outline in the LMS 400, is displayed to the students, the content suppliers and the tutors. The content proposal may be assessment questions, tutorial materials, videos or any other learning related materials to be provided through the LMS 400. The content proposal comprises a plurality of sub-portions. The plurality of sub-portions correlate entirely with the plurality of topics in the predetermined syllabus. Generally, the student will select the study material such that the entire syllabus is covered. In one embodiment, the content proposal comprises at least one of a plurality of study materials and a plurality of assessment questions. The content proposal may then go through a validation process to ensure that the content proposal complies with the syllabus outline within the LMS 400 and other conformity needed for the LMS 400 to function. For example, the LMS 400 may require various content-related-estimates. Each of the plurality of content-related-estimates comprises at least one of the difficulty level-estimate and the study time-estimate for one of the study materials. In other words, the plurality of content-related-estimates comprises a plurality of difficulty level-estimates and the plurality of study time-estimates for the plurality of study materials.

The content-related-estimates may be determined in several ways through the LMS 400. For example, the one or more content supplier may provide rough estimates, and the LMS 400 may adaptively adjust or improve the content-related-estimates as more data become available. Alternatively, the LMS 400 may put up the contents for trial uses and determine the estimates from a small sample data. In yet another embodiment, the content-related-estimates may be determined through rating administration component 450 and the prior use information processing component 470 by comparing the content proposal with a closest similar content stored in the database 410. The accuracy of the content-related-estimates at an initial stage may be optionally treated as “inaccurate” by the LMS 400 and the usage data at the initial stage may, optionally, disregarded for other purposes so as to reduce the impact of inaccuracy on the LMS 400. In a later step of the method 600, the content-related-estimates will be matched with user-related-estimates prior to be released for a plurality of students.

The user-related-estimates may be stored in the user-database 414. Prior to the matching process, the LMS 400 first determines the user-related-estimates by ensuring that the relevant data such as the proficiency level-estimate and the available time-estimate for the students are available in the user-database 414. In one embodiment, the plurality of user-related-estimates comprises at least one of a proficiency level-estimate and an available time-estimate for the student. Prior to matching with the content-related-estimates, the LMS 400 may optionally trigger the progress monitoring component 435, rating administration component 450, and prior use information processing component 470 to update the user-related-estimates.

The matching is primarily done through matching of two parameters, the time related component and the difficulty or proficiency level component. The time related component of the content proposal in total is less than the total available time. For example, the student who has a total of 40 hours per week should not be assigned with study materials that requires more than 40 hours to complete. The matching of the difficulty level-estimates of the content and the proficiency level-estimates of the students can be done in a few ways. A simple way is to assign a few grading levels, such as “normal”, “difficult”, and “easy” to the content proposals and to assign a few proficiency levels such as “average”, “higher” or “lower” to the students. Then the LMS 400 ensures that the students with proficiency level of average gets only those with average level. The example above with three-level grading may be suitable for LMS 400 with a scale of up to 100 students. For a higher number of students, the grading level should increase, for example, to ten-levels or more. The difficulty level-estimate of the study content matches the proficiency level-estimate of the student when the difficulty level-estimate and the proficiency level-estimate of the student differs less than a predetermined percentage margin. For avoidance of doubt, matching does not mean that the difficulty level-estimate and the proficiency level-estimate need to be exactly equal. For example, let's consider an example that the difficulty level-estimate of the study content, as well as the proficiency level-estimate, are categorized into 20 levels. A difficulty level-estimate of “9” may be considered as matching proficiency levels of “8”, “9”, and “10” if a tolerance of one level is accepted.

The proficiency level-estimates and the proficiency level-estimates may not stay fixed and may change under some conditions. The LMS 400 may adaptively adjust one or both the proficiency level-estimate and the available time-estimate of the student in order to improve the quality of matching. For example, the LMS 400 may adjust whenever one of the estimates is updated. Let's consider a first example where a first content of the content proposal, having a first content assessment question, is provided to a plurality of students through the LMS 400. When a first number of students or a first percentage of students, who have lower proficiency level-estimates compared to the difficulty level-estimate of the first content, answer correctly the first content assessment question, the difficulty level-estimate of the first content can be adjusted lower. Let's consider a second example where a second content of the content proposal, having a second assessment question, is provided to the students through LMS 400. When a second number of the plurality of students or a second percentage of students, who have higher proficiency level-estimates compared to the difficulty level-estimate of the second content, answer incorrectly the second content assessment question, the difficulty level-estimate of the second content can be adjusted higher. In a similar way, the study time-estimate of the content can be adjusted. For example, when more than a predetermined percentage of the plurality of students completed faster or slower than the study time-estimate of a specific assessment question by a predetermined margin, the study time-estimate can be adjusted lower or higher accordingly. For avoidance of doubt, the term “first” and “second” are used to distinguish the first and second example only and there is no sequential relationship. The LMS 400 adjusts the proficiency level-estimates and the study-time estimates when a threshold is met. Therefore, the first number of students, the second number of students and the predetermined percentage in the example above are usually derived from a threshold number calculated in terms of percentage. The threshold values are determined without considering individual students and may or may not include existing students who is using the LMS 400.

In another embodiment, the proficiency level-estimates and the proficiency level-estimates may comprise a rating from a rating system such as the Glicko Rating System, or the Elo Rating System or other rating system. The rating system is managed within the rating administration component 450 and a rating database 416. In general, a rating system is a method for calculating the relative skill levels of players in zero-sum games such as chess or any competitive sports. The rating system cannot be used as such and a minor tweak may be needed for the use in the LMS 400. Unlike sports or chess games, the students are not competing with each other, but solving assessment questions of the study materials. If the student solves a question correctly, the LMS 400 will consider that the student won the game. If the student solves the question incorrectly, the LMS 400 will consider that the “question” won the game. In this way, by assigning an initial rating to each student and assessment question, the ‘strength’ of the question and the student can be determined through the rating if sufficient usage takes place. For example, using the Elo rating system for chess, each question and each student will be assigned with a rating of 1500. Difficult questions or students with higher learning ability will obtain higher ratings of 2000 and above over time. Easy assessment questions or weaker students will end up having a rating below 1500. In this way, a correlation between the difficulty level of the assessment questions and the proficiency level of the students can be established.

In order to obtain a more accurate rating for new students or new assessment questions, the initial rating may be improved using other methods instead of starting with an initial rating number as in most rating systems. For example, prior to using the LMS 400, a student may provide his academic result outside the LMS 400 and an initial rating can be assigned lower or higher. Similarly, based on the experience of the content supplier, the rating of the assessment questions can be tweaked accordingly. If the LMS 400 adopts the rating system, the proficiency level-estimates of the students and the difficulty level-estimates of the contents may be the rating assigned through the rating administration 450. When the rating system is utilized, the proficiency level-estimates of the students and the difficulty level-estimates of the contents are considered matched if the difference is less than a predetermined margin percentage. For example, consider a case where the highest rating is 2500 and the lowest rating is 1000. The LMS 400 may decide that the difficulty level-estimate of the customized study content matches the proficiency level-estimate of a student when the difference is less than 10%, which is a rating of 150. In other words, the LMS 400 may assign assessment questions having a rating between 1850 and 2150 for a student who has a rating of 2000.

In summary, there are at least two matching parameters in the method 600, i.e. (1) matching of the difficulty level-estimate of the content proposal matches the proficiency level-estimate of the student, (2) matching of the study time-estimate the content proposal is less than the available time-estimate. There may be more parameters to be considered for matching purposes. For example, geographical location, school, age, and whether the student has sufficient time. Matching the proficiency level estimate does not mean that the proficiency level-estimate and the difficulty level-estimate has to be at the same level all the time. On the contrary, there can be a mixture. If the student is to be given only difficult questions to the extent that he can only do 10%, the confidence level will go down, and the student will be discouraged. On the contrary, if the students score almost 90% and above, the assessment questions are considered to be too easy for the student. The time spent on doing the questions is not efficient. A good mixture of assessment questions should be something that the students get 50%-80% correct. The assessment questions can be selected from a group where the rating or the grading level is higher (difficult questions) and from a second group where the rating or the grading level is lower (easy questions). If the student gets 90%, for example, then LMS 400 will select difficult questions for the student to answer until he gets lower mark. On the contrary, if the student gets 30% or lower, the LMS 400 will select easy questions until the mark improves to over 50%. In other words, the LMS 400 may adaptively select questions so that the student get a score that is between 50% and 80% for every practice session or tests so that the student will not end up spending all the time on something he knows, or having too many difficult questions that may destroy the self-confidence. The selection process for the next question may be conducted either by the content selection component 437 or the assessment test management component 438 when the student is answering question. The content selection component 437 and the assessment test management component 438 are instruction sets for selecting assessment questions. The content selection component 437 comprises instruction sets for selecting assessment questions or study material for learning in general. The assessment test management component 438 comprises instruction sets for selecting assessment questions for an assessment test. In one embodiment, the LMS 400 may comprise the content selection component 437 and not the assessment test management component 438 as the content selection component 437 will select assessment questions for all purposes.

The target of 50% and 80% may be adjusted based on the needs and character of the student. For example, the target may be adjusted lower to between 30% and 50% to ensure that the student ended up spending more time learning something. This may be done if the student is guided by a coach or there is a mean to confirm that having low scores will not destroy the student's self-confidence. Another situation where the target score should be adjusted lower is when the student is lack of study time or behind schedule. For example, when a study plan is established, a progress projection may be compiled by the scheduling component 434 and the target planning component 432. When an actual progress is received through the progress monitoring component 436, a comparison can be made between the actual progress and the progress projection to determine whether the student is ahead or behind schedule. When the student is behind schedule, the content selection component 437 may increase the proportion of difficult questions so that the student spends more time learning something he does not know and reduce time spent on something the student is anticipated to know already. One more step LMS 400 may take, when the student is behind schedule or significantly behind schedule, is to identify a portion of topics that are less popular or appear less frequently in the past examination paper. For example, not every topic from the syllabus will attract similar amount of assessment questions from the tutors or the content suppliers because such topics are less popular. In this way, the LMS 400 may have sufficient data to take out some study materials from such topics from the study plan so that the student may complete the syllabus within the targeted study period.

Assessment tests for the students may be selected from the assessment questions bank stored in the content database. The assessment tests may include practice test papers for practice and learning purposes. The difficulty level of the assessment test may be adjusted using the proficiency level-estimates of the students and the difficulty level-estimates of the contents to achieve specific goals outside education purpose. For example, the LMS 400 may include a welfare management component 439 that collects confidence level input from the parent or tutor or coaches through the tutor interface component 424. If a student is over-confident, a tutor or a mentor or a parent of the student may provide such input to the LMS 400 so that the assessment test management unit will generate more difficult questions for the student to get lower marks and avoid being over-confident. In other words, the assessment test can be set in such that the difficulty level may be made to correlate with the confidence-level-input so as to manage the student not to be overly confident, or to be lack of confidence. Similarly, for students with lower self-esteem, the questions can be made easier. The welfare management component 439 may also receive other health-related information from the tutor, parents or receive from an external device such as a smart watch or a pulse detector of the student. For example, a device for monitoring pulse rate may be connected to the LMS 400. The health-related information may comprise other information regarding the student such as the mental condition of the student, anxiety level, and other aspects of well-being of the students. If the student is overly anxious and have higher average pulse rate, the LMS 400 may be set to produce easier assessment tests or to reduce the available study time. This may be done with or without informing the student. For ordinary healthy student, the assessment test may be set to mix difficult questions and easy questions so that a targeted score is set between 50% and 80%. The target between 50% and 80% would ensure that the student does not spend all the time answering question that the student already know the answer. And yet, the score of more than 50% would ensure that the student will not be discouraged by difficult questions only.

Under conventional approach, the content suppliers are paid a sum whether or not the students use the material. In contrast, for the method 600, a new payment system where the student pays per usage will be used. The LMS 400 comprises a payment management component 446 to manage payment for the content suppliers. For the pay per use methodology, a payment process will be initiated when a student selects a content from a specific supplier. Initiating a payment process does not necessarily mean actual payment has been made. Initiating a payment process may comprise activities to calculate some form of payment which contribute to actual payment subsequently. For example, when a student selects an assessment question from a first supplier, the payment process may be initiated by recording the usage in the payment management component 446 for the first supplier. Similarly, when the student selects another assessment question from a second supplier, the usage will be recorded for the second supplier. The payment process may have been initiated but no actual payment had been transacted. The first and second suppliers will be paid when the total usage of all uses exceeds a predetermined payment quantity threshold. The student may pay a monthly or under a post-paid system to pay for a larger quantity of materials that may be created by different suppliers. In some embodiments, the fee paid by the student may include the fee for the tutors or coaches as well.

Additional aspects of the present disclosure contemplate a method for providing a customized study content to a student, the method comprising: (1) providing an LMS for maintaining a record of a content proposal, wherein the content proposal comprises at least one of a plurality of study materials and a plurality of assessment questions; (2) determining, with the LMS, a plurality of content-related-estimates, wherein the plurality of content-related-estimates comprise at least one of a difficulty level-estimate and a study time-estimate for the content proposal; (3) determining, with the LMS, a plurality of user-related-estimates, wherein the plurality of user-related-estimates comprises at least one of a proficiency level-estimate and an available time-estimate for the student; (4) determining a selected portion of the content proposal as the customized study content for the student; and (5) adaptively adjusting at least one of the plurality of content-related-estimates and the plurality of user-related-estimates.

Additional aspects of the present disclosure contemplate determining the selected portion of the content proposal as the customized study content for the student comprises matching the difficulty level-estimate of the selected portion of the content proposal with the proficiency level-estimate of the student.

Additional aspects of the present disclosure contemplate the selected portion of the content proposal are selected as the customized study content for the student if the study time-estimate of the selected portion of the content proposal is less than the available time-estimate.

Additional aspects of the present disclosure contemplate (1) a first selected content of the customized study content is selected from a first supplier; and (2) the method comprising initiating a payment process to the first supplier when the first selected content of the customized study content is used by the student.

Additional aspects of the present disclosure contemplate (1) the first selected content of the customized study content is selected by a plurality of additional students; and (2) paying the first supplier when a sum of unpaid uses arises from the student and the plurality of additional students become more than a predetermined payment quantity.

Additional aspects of the present disclosure contemplate the content proposal is submitted by one or more content-suppliers, and that the method further comprising requesting the one or more content suppliers to provide an initial estimate for the difficulty level-estimate and the study time-estimate of the content proposal respectively.

Additional aspects of the present disclosure contemplate the method further comprising adaptively adjusting at least one of the difficulty level-estimate and the study time-estimate of the plurality of study materials.

Additional aspects of the present disclosure contemplate (1) a first content of the content proposal is provided to a group of students, wherein the first content comprises a first content assessment question; (2) a first number of the group of students, who have lower proficiency level-estimates compared to the difficulty level-estimate of the first content, answer correctly the first content assessment question; and (3) the difficulty level-estimate of the first content is adjusted lower, when the first number is more than a first predetermined threshold.

Additional aspects of the present disclosure contemplate (1) a second content of the content proposal is provided to a group of students, wherein the second content comprises a second content assessment question; (2) a second number of the group of students, who have higher proficiency level-estimates compared to the difficulty level-estimate of the second content, answer incorrectly the second content assessment question; and (3) the difficulty level-estimate of the second content is adjusted higher when the second number is higher than a second predetermined threshold.

Additional aspects of the present disclosure contemplate (1) a third content of the plurality of study materials is provided to a group of students; and (2) the study time-estimate is adjusted when more than a predetermined percentage of the group of students completed faster or slower than the study time-estimate of the third content by a predetermined margin.

Additional aspects of the present disclosure contemplate the method further comprising adaptively adjusting at least one of the proficiency level-estimate and the available time-estimate of the student.

Additional aspects of the present disclosure contemplate the proficiency level-estimate is adjusted higher when the student answers correctly a predetermined amount of questions with a difficulty level-estimate higher than the proficiency level-estimate of the student.

Additional aspects of the present disclosure contemplate the available time-estimate for the student is updated periodically based on a progress of the student.

Additional aspects of the present disclosure contemplate the method further comprising collecting a health-related information about the student; and adjusting the available time-estimate for the student based on the health-related information.

Additional aspects of the present disclosure contemplate the method further comprising providing a practice test paper selected from the customized study content, wherein: (1) a first predetermined portion of the practice test paper is selected such that the difficulty level-estimate of the first predetermined portion of the practice test paper is higher than the proficiency level-estimate of the student; and (2) a second predetermined portion of the practice test paper is selected such that the difficulty level-estimate of the first predetermined portion of the practice test paper is lower than the proficiency level-estimate of the student.

Additional aspects of the present disclosure contemplate a ratio of the first predetermined portion and the second predetermined portion is adjusted in accordance with a personality input about the student that is related to self-esteem of the student.

Additional aspects of the present disclosure contemplate a substantial portion of the practice test paper is selected such that the proficiency level-estimate of the student is substantially equal to the difficulty level-estimate of the substantial portion of the practice test paper.

Additional aspects of the present disclosure contemplate the first predetermined portion and the second predetermined portion are adaptively selected such that the student scores in a range of 50%-80% in the practice test paper.

Additional aspects of the present disclosure contemplate the method further comprising: (1) obtaining an anxiety level information of the student; and (2) selecting, by using the LMS, the first predetermined portion and the second predetermined portion in accordance with the anxiety level information.

Additional aspects of the present disclosure contemplate the method further comprising: (1) providing a progress projection; and (2) generating an actual progress information based on a percentage of the customized study content completed by the student, wherein a ratio composition of the first predetermined portion and the second predetermined portion is adjusted based on a comparison between the progress projection and the actual progress information.

Additional aspects of the present disclosure contemplate the difficulty level-estimate of the customized study content matches the proficiency level-estimate of the student when the difficulty level-estimate and the proficiency level-estimate of the student differs less than a predetermined percentage margin.

Additional aspects of the present disclosure contemplate a learning management system (LMS) for providing a customized study content to a student, the LMS comprising at least one processor; and computer memory coupled to the at least one processor, wherein the computer memory comprises instructions that are executable by the at least one processor, and wherein the instructions comprise: (1) a content proposal record, wherein the content proposal record comprises at least one of a plurality of study materials and a plurality of assessment questions; (2) a content admin component configured to determine a plurality of content-related-estimates for the content proposal record, wherein the plurality of content-related-estimates comprise at least one of a difficulty level-estimate and a study time-estimate; (3) a user management component configured to determine a plurality of user-related-estimates for the student, wherein the plurality of user-related-estimates comprises at least one of a proficiency level-estimate and an available time-estimate for the student; and (4) a study plan admin component configured to determine a selected portion of the content proposal record as the customized study content for the student, wherein at least one of the plurality of content-related-estimates and the plurality of user-related-estimates are configured to be adaptively adjusted through one of the content admin component and the user management component respectively.

Additional aspects of the present disclosure contemplate the study plan admin component is configured to determine the selected portion of the content proposal record as the customized study content for the student such that the difficulty level-estimate of the selected portion of the content proposal record matches the proficiency level-estimate of the student.

Additional aspects of the present disclosure contemplate the study plan admin component is configured to determine the selected portion of the content proposal record as the customized study content for the student such that the study time-estimate of the selected portion of the content proposal record is less than the available time-estimate.

Additional aspects of the present disclosure contemplate the instructions further comprise a content supplier interface component and a payment component, wherein: (1) the content supplier interface component is configured to facilitate one or more content-suppliers to submit an additional portion of the content proposal record, and (2) a first selected content of the customized study content is selected from a first supplier of the one or more content suppliers; and (3) the payment component is configured to initiate a payment process to the first supplier when the first selected content of the customized study content is used by the student.

Additional aspects of the present disclosure contemplate both the difficulty level-estimate and the proficiency level-estimate comprises a common rating, and wherein the instructions further comprise a rating admin component to adaptively adjust at least one of the difficulty level-estimate and the proficiency level-estimate for each of the plurality of study materials.

Additional aspects of the present disclosure contemplate (1) a first content of the content proposal record is provided to a group of students, wherein the first content comprises a first content assessment question; (2) a first percentage of the group of students having lower proficiency level-estimates compared to the difficulty level-estimate of the first content, answer correctly the first content assessment question; and (3) the difficulty level-estimate of the first content is adjusted lower when the first percentage is higher than a first predetermined value.

Additional aspects of the present disclosure contemplate (1) a second content of the content proposal record is provided to a group of students wherein the second content comprises a second content assessment question; (2) a second percentage of the group of students having higher proficiency level-estimates compared to the difficulty level-estimate of the second content, answer incorrectly the second content assessment question; and (3) the difficulty level-estimate of the second content is adjusted higher when the second percentage is higher than a second predetermined value.

Additional aspects of the present disclosure contemplate the instructions further comprise a scheduling component, wherein: (1) a third content of the plurality of study materials is provided to a plurality of additional students in addition to the student; and (2) the scheduling component is configured to adjust the study time-estimate when more than a predetermined percentage of the plurality of additional students completed faster or slower than the study time-estimate of the third content by a predetermined margin.

Additional aspects of the present disclosure contemplate the user management component is configured to adaptively adjust at least one of the proficiency level-estimate and the available time-estimate of the student.

Additional aspects of the present disclosure contemplate the instructions further comprise a welfare management component configured to collect a health-related information, and wherein the user management component is configured to adjust the available time-estimate for the student based on the health-related information.

Additional aspects of the present disclosure contemplate the welfare management component is coupled to a pulse rate tracking device that tracks the pulse rate of the student, and the welfare management component is configured to determine whether the student is in an anxious state based on the pulse rate.

Additional aspects of the present disclosure contemplate the welfare management component is configured to receive a confidence-level-input about the student from a coach or a tutor of the student.

Additional aspects of the present disclosure contemplate the instructions further comprises an assessment test management component configured to generate an assessment test for the student, and wherein the assessment test has a difficulty level that correlates with the confidence-level-input.

Additional aspects of the present disclosure contemplate the instructions further comprise a content selection component configured to select a set of assessment questions to be set as a practice test for the student from the content proposal record, wherein the set of assessment questions have difficulty level-estimates respectively.

Additional aspects of the present disclosure contemplate (1) the content selection component is configured to set a first predetermined portion of the practice test such that the difficulty level-estimates of the assessment questions are higher than the proficiency level-estimate of the student; and (2) the content selection component is configured to set a second predetermined portion of the practice test such that the difficulty level-estimates of is the assessment questions are lower than the proficiency level-estimate of the student.

Additional aspects of the present disclosure contemplate the content selection component is configured to adaptively select the assessment questions from the first predetermined portion and the second predetermined portion are such that the student scores in a range of 50%-80% in the practice test.

Additional aspects of the present disclosure contemplate the content selection component is configured to increase the first predetermined portion and decrease the second predetermined portion when the user management component receive a request to reduce a total study time from the student.

Additional aspects of the present disclosure contemplate the content selection component is configured to increase the first predetermined portion and decrease the second predetermined portion when the user management component receive a first input from a coach that the student is over-confident.

Additional aspects of the present disclosure contemplate the content selection component is configured to decrease the first predetermined portion and increase the second predetermined portion when the user management component receive a second input from a coach that the student is overly anxious.

Additional aspects of the present disclosure contemplate a computer system for providing a LMS to a student, the student is one student from a group of students using the LMS, the computer system comprising a memory and at least one processor coupled to the memory, the at least one processor is configured to: (1) store a content proposal record, wherein the content proposal record comprises a plurality of assessment questions; (2) determine a plurality of content-related-estimates, wherein each of the plurality of content-related-estimates comprises a difficulty level-estimate and a study time-estimate; (3) determine a plurality of user-related-estimates, wherein each of the plurality of user-related-estimates comprises a proficiency level-estimate and an available time-estimate for the student; (4) adaptively update the plurality of user-related-estimates and the plurality of content-related-estimates; and (5) select a customized study content for the student by matching the plurality of user-related-estimates and the plurality of content-related-estimates that had been updated.

Additional aspects of the present disclosure contemplate the at least one processor is further configured to maintain the difficulty level-estimate and the proficiency level-estimate based on a rating system.

Additional aspects of the present disclosure contemplate the at least one processor is further configured to display a prior use data to the student for selecting the customized study content.

Additional aspects of the present disclosure contemplate the at least one processor is further configured to: (1) maintain a study plan record representing a time schedule of the student to complete the customized study content; (2) collect a record representing a progress information of the student; (3) compute a deviation calculation between the progress information and the study plan record; and (4) adjust the study time-estimate based on the deviation calculation.

Additional aspects of the present disclosure contemplate the at least one processor is further configured to increase the difficulty level-estimate of a first assessment question when a predetermined number of students, who have a proficiency level-estimate that is higher than the difficulty level-estimate of the first assessment question, answer incorrectly the first assessment question.

Additional aspects of the present disclosure contemplate the at least one processor is further configured to decrease the difficulty level-estimate of a second assessment question when a predetermined number of students, who have a proficiency level-estimate that is lower than the difficulty level-estimate of the second assessment question, answer correctly the second assessment question.

Additional aspects of the present disclosure contemplate the at least one processor is further configured to increase the proficiency level-estimate of the student when the student answers correctly a predetermined number of assessment questions which have difficulty level-estimates that are higher than the proficiency level-estimate of the student.

Additional aspects of the present disclosure contemplate the at least one processor is further configured to decrease the proficiency level-estimate of the student when the student answers incorrectly a predetermined number of assessment questions which have difficulty level-estimates that are lower than the proficiency level-estimate of the student.

Additional aspects of the present disclosure contemplate the at least one processor is further configured to adjust the study time-estimate of a third assessment question when a predetermined number of students answer faster or slower than the study time-estimate by a predetermined margin.

Additional aspects of the present disclosure contemplate the at least one processor is further configured to: (1) store a record of difficult questions having difficulty-level estimates that are higher than the proficiency level-estimate of the student; (2) store a record of easy questions having difficulty-level estimates that are lower than the proficiency level-estimate of the student; and (3) select the customized study content such that the customized study content comprises a difficult portion of questions selected from the record of difficult questions, and an easy portion of questions selected from the record of easy questions.

Additional aspects of the present disclosure contemplate the at least one processor is further configured to select the customized study content such that more than 50% of the customized study content comprises the difficult portion of questions.

Additional aspects of the present disclosure contemplate the at least one processor is further configured to adaptively select the customized study content from the difficult portion of questions and the easy portion of questions such that the student answers correctly, between 50% and 80%, the assessment questions from the customized study content.

Additional aspects of the present disclosure contemplate the at least one processor is further configured to adjust a ratio of the difficult portion of questions and the easy portion of questions based on a health-related input from a tutor, and wherein the health-related input comprises at least one of anxiety level information and confidence level information.

Additional aspects of the present disclosure contemplate the at least one processor is coupled to a health monitoring device, wherein the at least one processor is further configured to adjust a ratio of the difficult portion of questions and the easy portion of questions based on an input from the health monitoring device.

Additional aspects of the present disclosure contemplate the at least one processor is further configured to increase the difficult portion of questions in the customized study content when the student is behind schedule.

### III Prior Use Data and its Utilization

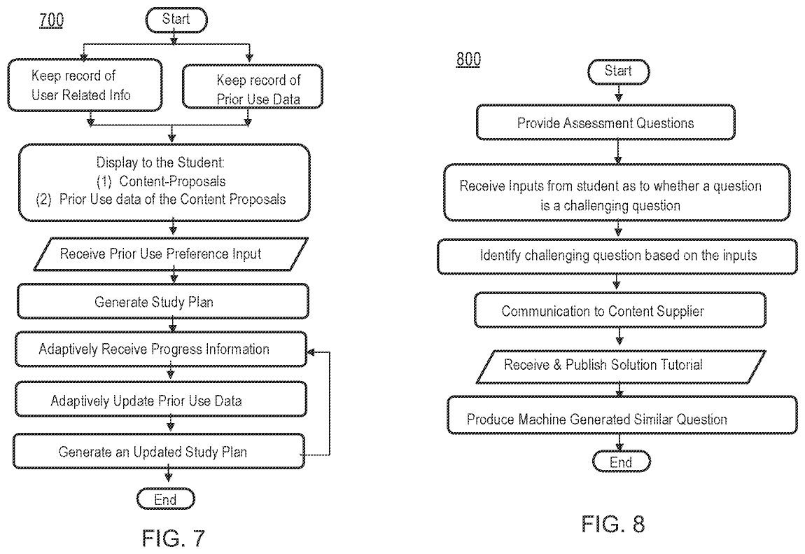

FIG. 7 shows a method 700 that utilizes prior use data to improve efficiency of a study plan. As explained above, the LMS 400 and the content are used by a plurality of students. A student studying a subject matter should be able to look at the data from other students who learned the same subject. The prior use data may include usage for each of the study materials, ratings, how well other students did after using the study material, and any other information that may help the student. A top student who did well in the examination would be interested to look at what other top students did previously and not what other weaker students use to study for the same examination because the material used by a weaker student is likely too easy tor the top student. Using the prior use data will enable the student to select suitable study material as illustrated in the method 700. The method 700 starts by maintaining a record that comprises a plurality of user-related information such as the proficiency level-estimates, available time-estimates and other parameters needed to the LMS 400 to search from the prior use data. For a new user, maintaining a record comprises receiving the plurality of user-related information from the new user. The method 700 also includes maintaining a record of prior use data. If the prior use data does not require updates, maintaining a record comprises storing the record in the memory of the LMS 400. The prior use data is the information derived from previous use of the content proposal by the students. The prior use data of an assessment question may comprise various information related to the assessment question such as popularity, whether students who previously attempted the question find the question difficult, time used to attempt the assessment question, whether the students who managed to answer the assessment question obtain a satisfactory grade, and any other aspects that is beneficial to students who will attempt the assessment subsequently. For example, the prior use data may comprise a prior-user proficiency level estimate for the plurality of students who used the LMS in the previous academic year or the three previous academic years. In one embodiment, the prior use data may comprise a usage statistic histogram illustrating how many students used each of the content proposal. In another embodiment, the prior use data may comprise a review information about the content suppliers, or a review information about the content proposals. For example, the user may provide a popularity vote on satisfaction for the content suppliers as well as the study materials generated. In yet another embodiment, the prior use data may comprise a rating from rating systems such as Elo rating system, Glicko rating system or other equivalent rating system. A rating is updated, and it evolved after every use previously by other students. The rating of a question itself comprises a substantial information of the prior usage. For example, a higher rating (for example 2200) of an assessment question is attributed to the frequency of uses because all ratings start at a lower number (1500) or because other users find the assessment question too difficult. The prior use data may potentially improve the study time-estimates. For example, the prior use data may comprise a histogram illustrating actual time used by other students in previous year showing a distribution of time needed for each of the content proposal. After attempting some of the content proposals, the student would be able to know which group in the distribution he belongs. In this way, the study time-estimates can be improved.

Next, the method proceeds to the next step to display to the student a few content-proposals together with the prior use data which had been compiled. The prior use of the content-proposals comprises difficulty level-estimates, study time-estimates, histogram and other relevant statistics which may help the student to select or decide on which content-proposals are suitable. Optionally, in order to narrow down to a selected content, a preference-input with regard to the prior use data can be provided. In one embodiment, the preference-input may be related to the proficiency level of prior students who used the LMS 400 in previous year. For example, a student who aims to be in top 10% would focus on prior use data of other students who scored and made it to the top 10%. The student may also select other preference inputs related to the content-suppliers of choice, syllabus, range of difficulty levels, geographical location of the prior students and any other ways that may influence the selection of the content-proposals. By using the preference inputs as a filter, the LMS 400 would be able to narrow down and locate more suitable contents and compute a more suitable study plan for the student. FIG. 2B shows an example of how a filter is used to narrow down the choices for the content proposals. The method 700 then proceeds to generate a study plan based on the prior use data and the plurality of user-related information. If the preference input is provided, the preference input may also be considered when generating the study plan. The preference input may be applied indirectly such that the preference input filters out a portion of the prior use data.

Next, the method 700 proceeds to the updating phase whereby the LMS 400 keeps updating progress information and the prior use data. The updating phase will improve the study plan further. For example, students in previous academic years may be weak at a specific topic of the syllabus, but the tutors and student in the current year may have gone through more lessons on the specific topic. The prior use data that students are weak on the specific topic may be no longer accurate. After a period of time, new data collected in the prior use data may reflect more reality because the new data are attributed by use of students in the current academic year. In addition, the progress information would also reflect that students in the current year do not have issue on the specific topic of the syllabus. The method 700 may comprise the step of generating an updated study plan that is based on the updated version of prior use data. For example, the difficulty level-estimates of the content proposals maybe updated using a new data collected from the same fiscal year, and the improved study plan may be generated using the current fiscal year data. Similarly, the study time-estimates of the content proposals maybe updated using the data from the current fiscal year and the study plan may be updated solely based on data from the current fiscal year.

Additional aspects of the present disclosure contemplate a method for providing a customized study content to a student by way of an LMS, the method comprising: (1) maintaining a user record that comprises a plurality of user-related information of the student, wherein the user-related information comprises a proficiency level-estimate and an available time-estimate; (2) communicating, to the student, a plurality of content-proposals from one or more content suppliers, wherein the plurality of content-proposals comprises difficulty level-estimates and study time-estimates; (3) maintaining a usage record that comprises prior-use-data by a plurality of additional students related to the plurality of content-proposals, wherein the prior-use-data comprises usage statistics of the plurality of additional students; and (4) generating a study plan for the student based on the prior-use-data, and the plurality of user-related information.

Additional aspects of the present disclosure contemplate the method further comprising: (1) periodically updating the prior-use-data; and (2) generating an improved study plan based on the prior-use-data that has been updated.

Additional aspects of the present disclosure contemplate the method further comprising: (1) updating the difficulty level-estimates of the plurality of content-proposals based on a first updated version of the prior-use-data; and (2) generating an improved study plan based on the difficulty level-estimates that have been updated.

Additional aspects of the present disclosure contemplate the method further comprising: (1) updating the study time-estimates of the plurality of content-proposals based on a second updated version of the prior-use-data; and (2) generating an improved study plan based on the study time-estimates that have been updated.

Additional aspects of the present disclosure contemplate the prior-use-data comprises a prior-user-proficiency-level-estimate for the plurality of additional students.

Additional aspects of the present disclosure contemplate the prior-use-data comprises a histogram illustrating the prior-user-proficiency-level-estimate of the plurality of additional students.

Additional aspects of the present disclosure contemplate each of the plurality of additional students spent a first amount of time on a first content of the plurality of content-proposals, and wherein the prior-use-data related to the first content comprises statistics related to the first amount of time for each of the plurality of additional students.

Additional aspects of the present disclosure contemplate the prior-use-data comprises a review information about the one or more content suppliers.

Additional aspects of the present disclosure contemplate the prior-use-data comprises a review information about the plurality of content-proposals by one of the plurality of additional students.

Additional aspects of the present disclosure contemplate the prior-use-data is sorted according to geographical location of the plurality of additional students.

Additional aspects of the present disclosure contemplate the method further comprising generating a user ranking for the student, wherein one factor for deciding the user ranking is by a percentage of time the student has studied as compared to an original plan.

Additional aspects of the present disclosure contemplate (1) the plurality of content-proposals comprises a plurality of assessment questions, and a plurality of solution tutorials corresponding to the plurality of assessment questions; (2) a first content supplier of the one or more content suppliers provides a first assessment question, and a first solution tutorial corresponding to the first assessment question; (3) a second content supplier of the one or more content suppliers provides a second solution tutorial corresponding to the first assessment question; and (4) providing, to the student, a piece of prior-use-data on how well the plurality of additional students who selected each of the first solution tutorial and the second solution tutorial did in a past examination as a selection guide.

Additional aspects of the present disclosure contemplate the method further comprising receiving, from the student, a preference-input in response to the prior-use-data.

Additional aspects of the present disclosure contemplate the preference-input comprises at least one preference as to which one of the one or more content suppliers is preferred by the student.

Additional aspects of the present disclosure contemplate the preference-input comprises at least one preference as to difficulty levels of the study plan the student is willing to accept.

Additional aspects of the present disclosure contemplate the preference-input comprises at least one preference as to whether he prefers a first plan used by a first group of prior students who obtained higher grades, or a second plan that is used by a second group of prior students who obtained average grades.

Additional aspects of the present disclosure contemplate a learning management system (LMS) for providing a customized study content to a student, the LMS comprising: at least one processor; and computer memory coupled to the processor, wherein the computer memory comprises instructions that are executable by the processor, and wherein the instructions comprise: (1) a student interface component for the student to provide a plurality of user-related information, wherein the user-related information comprises a proficiency level-estimate and an available time-estimate; (2) a content selection component configured to provide a plurality of content-proposals from one or more content suppliers as selection candidates, wherein the plurality of content-proposals comprises difficulty level-estimates and study time-estimates; (3) a prior use information processing component configured to provide prior-use-data to the student, wherein the prior-use-data is taken when a plurality of additional students, who has a plurality of prior-user-related information, selected the plurality of content-proposals, and wherein the prior-use-data comprises usage statistics of the plurality of additional students; (4) a study plan administration component configured to receive, from the student, a preference-input in response to the prior-use-data; and (5) a scheduling component configured to generate a study plan for the student based on at least the preference-input, the prior-use-data, and the plurality of user-related information.

Additional aspects of the present disclosure contemplate the prior use information processing component is configured to periodically update the prior-use-data, and wherein the scheduling component is configured to generate an improved study plan based on the prior-use-data that has been updated.

Additional aspects of the present disclosure contemplate a content administration component configured to update the difficulty level-estimates of the plurality of content-proposals after the prior-use-data is updated, and wherein the scheduling component is configured to generate an improved study plan based on the difficulty level-estimates that have been updated.

Additional aspects of the present disclosure contemplate a content administration component configured to update the study time-estimates of the plurality of content-proposals after the prior-use-data is updated, and wherein the scheduling component is configured to generate an improved study plan based on the study time-estimates that have been updated.

Additional aspects of the present disclosure contemplate the prior-use-data comprises a usage statistic generated by the prior use information processing component based on data from the plurality of additional students.

Additional aspects of the present disclosure contemplate (1) the plurality of content-proposals comprises a plurality of assessment questions, and a plurality of solution tutorials corresponding to the plurality of assessment questions; (2) a first content supplier of the one or more content suppliers provides a first assessment question, and a first solution tutorial corresponding to the first assessment question; (3) a second content supplier of the one or more content suppliers provides a second solution tutorial corresponding to the first assessment question; (4) the student interface component is configured to provide, for selection purpose, the first solution tutorial and the second solution tutorial as choices for the student to select a solution tutorial; and (5) the prior use information processing component is configured to generate a popularity data for each of the first solution tutorial and the second solution tutorial as the prior-use-data.

Additional aspects of the present disclosure contemplate computer system for providing an LMS to a student, the computer system comprising a memory and at least one processor coupled to the memory, the at least one processor is configured to: (1) store a user record comprising a plurality of user-related information from a plurality of students of the LMS, wherein the plurality of the user-related information comprises a proficiency level-estimates and an available time-estimates; (2) store a study material record comprising a plurality of content-proposals from one or more content suppliers, wherein the plurality of content-proposals comprises difficulty level-estimates and study time-estimates; (3) store a prior-use-record for the plurality of content-proposals based on a usage by the plurality of students, wherein the prior-use-record for the plurality of content-proposals comprises information related to usage frequency, proficiency level-estimates of the plurality of students at a time when the plurality of students attempted the plurality of content-proposals, and an indication of time needed by the plurality of students took to complete plurality of content-proposals; and (4) select a customized study content for the student based on at least one of the prior-use-record, the difficulty level-estimates, the study time-estimates, the proficiency level-estimates and the available time-estimates.

Additional aspects of the present disclosure contemplate the at least one processor is further configured to generate a study plan for the student based on at least one of the prior-use-record, the difficulty level-estimates, the study time-estimates, the proficiency level-estimates and the available time-estimates, and wherein the study plan includes a time schedule for the student to complete the customized study content.

Additional aspects of the present disclosure contemplate the at least one processor is further configured to periodically update the prior-use-record; and to generate an improved study plan based on the prior-use-record that has been updated.

Additional aspects of the present disclosure contemplate the prior-use-record comprises a histogram illustrating the proficiency level-estimates of the plurality of students.

Additional aspects of the present disclosure contemplate each of the plurality of students spent a first amount of time on a first content of the plurality of content-proposals, and wherein the prior-use-record comprises a histogram illustrating the first amount of time of the plurality of students.

Additional aspects of the present disclosure contemplate the at least one processor is further configured to receive from the student, a preference-input in response to the prior-use-record; and the customized study content for the student is selected according to the preference-input.

Additional aspects of the present disclosure contemplate the preference-input comprises a preference to filter out prior-use-data of a specific group of students.

Additional aspects of the present disclosure contemplate wherein the preference-input comprises at least one preference as to whether he prefers a first plan used by a first group of students who obtained higher grades, or a second plan that is used by a second group of students who obtained average grades.

### IV Solution Tutorial Generation

Towards the end of a learning journey, an assessment test or an examination will be held. The examination usually comprises a plurality of assessment questions to test the understanding of the students on the topics learned. In most classes, a tutorial usually includes tutorials on how to solve those assessment questions, especially a more representative ones or more difficult ones. Tutorials can be done online, the solution tutorial may be provided in a video format, either as a standalone solution tutorial, or a part of a lesson. An experience teacher would know how to deliver a solution tutorial that meets the need of the majority of students, but it is almost impossible to meet the needs of all students in the classroom. There needs to have a method to guide the tutors or the teachers to produce solution tutorials in a more efficient manner. FIG. 8 shows a method 800 for generating a solution tutorial. The method 800 starts with the step of providing a plurality of assessment questions to a plurality of students. The assessment questions may be provided electronically through the LMS 400, or in a paper based forms. Similar to previous embodiments, the plurality of assessment questions may be questions provided by one or more content suppliers. In some embodiment, the plurality of assessment questions may also include past examination questions. When the students attempt the assessment questions, each of the students may not understand or may not know how to solve some of the assessment questions, but usually not all of the assessment questions. Therefore, the tutors do not need to produce solution tutorials for all of the assessment questions. However, the tutors may not be able to identify which assessment questions need a solution tutorial most. Potentially, the assessment questions that many students answer incorrectly may be a potential challenging assessment question for the student. However, some students may not know the answer and yet, guess correctly the answer. One way to capture such questions would be to allow the students to mark the question that they do not know the answer as “not sure” or “don't know”. This category of questions are the questions that the students have to guess the solution and not entirely sure about the solution. In other words, questions marked as “not sure” or “don't know” are potential challenging questions. A challenging question is an assessment that requires further explanation, guidance from a tutor or someone who coach the student. In this embodiment, the term challenging assessment question includes questions that a number of students find difficult, answered incorrectly, or needs further explanation.

The method 800 next proceeds to receiving inputs from the students regarding whether a question is a challenging question. As explained above, one way to receive inputs is to collect data on the number of students who answered a question incorrectly. In one embodiment, the LMS 400 includes an interface to allow a student to flag the assessment question as “don't′ know” for the questions that the student does not know the answer. The student may mark the question as “not sure” when the student answers a set of assessment question and not after completing the test when the student reviews the answer. This interface allows the student to capture the question that he finds challenging, but he may make a guess and eventually get a right answer although the student does not know the answer. The inputs from the students are obtained when the students answered a question incorrectly or marked a question as “not sure”. Alternatively, the student may make a submission that an assessment question should be categorized as a challenging assessment question. In other words, the inputs from the students includes an answer by the students for the specific assessment question, or a marking on the assessment question, or a direct request to categorize the assessment question as a challenging assessment question, or any other inputs that indicate whether a question should be categorized as a challenging assessment question. When the number of inputs exceeds a predetermined number, the method 800 then proceeds to identify the question as a challenging question. In some embodiments, the process of identifying the question as a challenging question may be done adaptively. In other words, the process of identifying may be initiated as soon as an input from the student is received. Alternatively, the process of identifying may be carried out at a predetermined short intervals. In this way, the tutors will be able to provide the solution tutorial similar to an on-demand basis. Optionally, the method 800 may comprise receiving a request from the students to identify the challenging assessment questions from the plurality of assessment questions.

The predetermined number of students may be one or more students. For example, let's consider an LMS 400 that has a first assessment question and a second assessment question. About one hundred students answered the first assessment question incorrectly or marked as “not sure”, whereas about five students answered the second assessment question incorrectly or marked as “not sure”. The first assessment question is a better candidate for the tutors to provide a solution tutorial as compared to the second question because the tutors goal is to provide tutorial lessons for questions that the majority of students do not know the answer. The LMS 400 may have a predetermined threshold of 10% or 100 students, which will end up identifying the first assessment question as challenging but not the second assessment question. Optionally, the predetermined threshold can be as low as one student for some circumstances, where there are more ways to analyze the data or there are circumstances that setting the low threshold will not end up having too many questions categorized as a challenging question. For example, the tutors may be interested in the inputs of a specific group of students, such as someone affiliated to the same organization or the student under his care. In such circumstances, the LMS 400 may comprise an interface the tutor to analyze or sieve the information that the tutor needs to create the solution tutorial. For example, the LMS 400 may provide a filtering interface for the tutor to sieve the data so as to compute a list of challenging assessment question for a predetermined group of students. Optionally, the tutor interface component of the LMS 400 is configured to provide a ranking list of challenging assessment questions as perceived by the plurality of students from a pre-selected group, without interference of the tutor. For example, the tutor interface may be configured to pre-select students for the tutor by using other information such as geographical information, location, schools of the students and the tutors.

Next, the LMS 400, through the tutor interface 424, communicate the challenging assessment questions to the plurality of tutors so that the tutor can access the LMS 400 to view the challenging assessment questions and subsequently produce solution tutorials. The solution tutorials may include lessons, material, videos or any other materials to assist a student to find an answer or to resolve an assessment question, or a topic in which the assessment question is derived from. The solution tutorials may be in a video format explaining the answer, or how the challenging assessment question can be resolved or answered. The solution tutorial may also include explanation in text form that shows the students how to obtain the correct answer or solution to an assessment question. Communicating the assessment questions includes notifying the tutors about assessment questions that require further material to assist understanding. For example, the LMS 400 may communicate the challenging assessment question by sending over a list of questions to the tutors, or to put the list of questions on a website. In other embodiment, the LMS 400 does not explicitly terms an assessment questions as challenging but communicate the challenging assessment questions by publishing or sending a histogram or a ranking list of the assessment question indicating which question has the most number of incorrect attempts. In yet another embodiment, the LMS 400 summarizes and produces a report to the tutors who indicates preferences in specific topics or specific areas in their expertise. The method 800 proceeds to the step of receiving the solution tutorials created by the tutors and then publish the solution tutorials for the students to access.

In addition to the tutors, the LMS 400 may optionally provide other students or content suppliers the list of challenging questions so that the students and the content suppliers may have a chance to produce a solution tutorial. By doing this, the LMS 400 considers another potential role of other students or content suppliers as a tutor. As explained in previous embodiment, the LMS 400 adopts a pay per use methodology. The students who know the answer of challenging assessment questions would have a chance to submit a solution tutorial so as to obtain some income. On some occasions, more than one solution tutorial may have been created by one or more parties, such as from one of the plurality of content-suppliers, the plurality of students and the plurality of tutors. The plurality of solution tutorials for one assessment question may be displayed to the students together with some prior use data. The prior use data may include comments by others which may be referred to by other students when they are deciding which solution tutorial to use. In one embodiment where a plurality of solution tutorials and a plurality of similar questions are provided for a predetermined challenging assessment question, the LMS 400 is configured to display the plurality of solution tutorials and the plurality of similar questions as well as the prior use data on a screen so that the student has all the information at one location.

In addition to the solution tutorial, the students, the tutors and the content suppliers can also help to produce a similar question that tests a similar concept as compared to the challenging assessment question. The similar question allows the students to practice on the same subject matter, which is one effective way to provide deliberate practice. The similar question may be pre-generated by human and selected by the LMS. FIG. 9 shows examples of similar questions. For the subject of Mathematic, generating a similar question may mean replacing the numerical numbers with a new set of numbers as shown in FIG. 9. For the subject of English, generating a similar question for a grammar question may be testing the same aspect of the grammar such as tenses. In the example shown in FIG. 9, the concept tested is to use the correct tenses. Both assessment questions require the student to use the correct tense for “does” in the sentence. Referring to the example for the subject of Science shown in FIG. 9, the concept tested is the requirement of the exposure to light for photosynthesis to take place for both assessment questions. The similar assessment questions are effective for revision purposes. The questions look dissimilar but testing the same aspect. If the student does not understand or attempt to ‘memorize’ the answer, the student may not be able to answer all the similar questions. On some occasion, the similar questions can be automatically generated by the LMS 400. In other words, the LMS 400 may produce a machine generated similar question for the student. For example, for the subject of Mathematics, the LMS 400 may machine read the numbers and regenerate the numerical number automatically to produce the similar assessment questions. For the subject of English, the LMS 400 may perform a search from the website to find similar sentences so as to produce a similar question. An easier way to generate the similar question will be to replace the nouns, and to rewrite the whole sentence. Other technique to create the similar question may be using natural language processing technique. The LMS 400 may optionally remind the students who did a challenging assessment question incorrectly to attempt again the challenging assessment question or the similar question. The challenging question as well as the similar questions should be tested at a predetermined time intervals to ensure that the student has fully understood the concept. For example, the challenging question and the similar question can be shown to the student again after one day, one week, two weeks, and one month. For example, the study plan admin component 430 may be configured to select similar question for the student to practice at intervals at incremental basis.

Additional aspects of the present disclosure contemplate a method for providing a solution tutorial, by using an LMS, the method comprising: (1) providing, through the LMS, a plurality of assessment questions for a plurality of students; (2) receiving inputs, from the plurality of students, about whether a question of the plurality of assessment questions is a challenging assessment question; (3) identifying, by way of the LMS, the question as a challenging assessment question when a number of inputs from the plurality of students exceeds a predetermined threshold; (4) communicating, to a plurality of tutors, about the challenging assessment question; and (5) receiving, from the plurality of tutors, at least one solution tutorial with respect to the challenging assessment question.

Additional aspects of the present disclosure contemplate the method further comprising providing an interface for the plurality of students to flag at least one of the plurality of assessment questions as “not-sure” when answering the plurality of assessment questions.

Additional aspects of the present disclosure contemplate the plurality of students provides the inputs by answering incorrectly the plurality of assessment questions or by marking the plurality of assessment questions as “not sure”.

Additional aspects of the present disclosure contemplate the method further comprising receiving a submission from one of the plurality of students, wherein the submission comprises a request to mark one of the plurality of assessment questions as the challenging assessment question.

Additional aspects of the present disclosure contemplate (1) the LMS identifies a first challenging assessment question; and (2) the method further comprising receiving from one of a plurality of content-suppliers, the plurality of students and the plurality of tutors a first solution tutorial with respect to the first challenging assessment question.

Additional aspects of the present disclosure contemplate the method further comprising: (1) receiving from at least one of the plurality of content-suppliers, the plurality of students and the plurality of tutors an additional solution tutorial with respect to the first challenging assessment question; and (2) providing prior use data for the plurality of students to select between the first solution tutorial and the additional solution tutorial.

Additional aspects of the present disclosure contemplate (1) the LMS identifies a second challenging assessment question; and (2) the method further comprising receiving from, one of a plurality of content-suppliers, the plurality of students and the plurality of tutors, a similar second question with regard to the second challenging assessment question testing a similar aspect as the second challenging assessment question.

Additional aspects of the present disclosure contemplate the method further comprising reminding the plurality of students who answered the second challenging assessment question incorrectly to attempt the similar second question after a predetermined time interval.

Additional aspects of the present disclosure contemplate the LMS identifies a third challenging assessment question, and wherein the method further comprising providing a machine-generated similar third question with regard to the third challenging assessment question testing a similar aspect as the third challenging assessment question.

Additional aspects of the present disclosure contemplate the third challenging assessment question is a mathematical related problem, and wherein the machine-generated similar third question is substantially identical but numerical numbers are changed.

Additional aspects of the present disclosure contemplate an LMS, comprising: at least one processor and computer memory coupled to the processor, wherein the computer memory comprises instructions that are executable by the processor, and wherein the instructions comprise (1) a study material record comprising a plurality of assessment questions into the LMS; (2) a student interface component configured to allow a plurality of students to access the plurality of assessment questions; (3) a content admin component configured to identify at least one of the plurality of assessment questions as a challenging assessment question; and (4) a tutor interface component configured to communicate to a plurality of tutors about the challenging assessment question, and to allow the plurality of tutors to submit at least one solution tutorial with respect to the assessment question.

Additional aspects of the present disclosure contemplate the student interface component is configured to allow the plurality of students to mark a question as “not sure” when answering the plurality of assessment questions.

Additional aspects of the present disclosure contemplate one assessment question was answered incorrectly or marked as “not sure” by more than a predetermined number of students, and wherein the content admin component is configured to identify the one assessment question as the challenging assessment question.

Additional aspects of the present disclosure contemplate that the LMS further comprising a content-supplier interface component configured to allow a plurality of content suppliers to submit an additional portion of the plurality of assessment questions, and wherein (1) a first challenging assessment question is identified by the content admin component; and (2) the LMS is configured to allow at least one of the plurality of content-suppliers, the plurality of students and the plurality of tutors to submit a first solution tutorial with respect to the first challenging assessment question.

Additional aspects of the present disclosure contemplate that the LMS further comprising a content-supplier interface component configured to allow a plurality of content suppliers to submit an additional portion of the plurality of assessment questions, and wherein (1) a second challenging assessment question is identified by the content admin component; and (2) the LMS is configured to allow at least one of the plurality of content-suppliers, the plurality of students and the plurality of tutors to submit a similar second question with regard to the second challenging assessment question testing a similar aspect as the second challenging assessment question.

Additional aspects of the present disclosure contemplate (1) a third challenging assessment question is identified by the content admin component; and (2) the LMS comprises a content administration unit configured to generate a machine-generated similar third question with regard to the third challenging assessment question testing a similar aspect as the third challenging assessment question.

Additional aspects of the present disclosure contemplate the student interface component is configured to allow the plurality of students to make a request to identify an assessment question as the challenging assessment question.

Additional aspects of the present disclosure contemplate a computer system for a solution tutorial, the computer system comprising a memory and at least one processor coupled to the memory, the at least one processor is configured to (1) store a study material record comprising a plurality of assessment questions; (2) allow a plurality of students access, to the plurality of assessment questions, so that the plurality of students attempt to solve at least one assessment question of the plurality of assessment questions; (3) store a result record for the at least one assessment question that comprises results of the plurality of students; (4) identify the at least one assessment question of the plurality of assessment questions as a challenging assessment question based on the results of the plurality of students; (5) communicate to a plurality of tutors about the challenging assessment question; and (6) receive from at least one of the plurality of tutors a solution tutorial for the challenging assessment question.

Additional aspects of the present disclosure contemplate the at least one processor is further configured to identify the at least one assessment question as the challenging assessment question if more than a predetermined number of students from the plurality of students answered incorrectly.

Additional aspects of the present disclosure contemplate the at least one processor is further configured to identify the at least one assessment question as the challenging assessment question if more than a predetermined number of students from the plurality of students marked the at least one assessment question as “not sure”.

Additional aspects of the present disclosure contemplate the at least one processor is further configured to identify the at least one assessment question as the challenging assessment question if more than a predetermined number of students from the plurality of students make a request to assign the at least one assessment question as the challenging assessment question.

Additional aspects of the present disclosure contemplate the at least one processor is further configured to (1) store a record of a set of challenging assessment questions; (2) communicate the set of challenging assessment questions to one of a plurality of content-suppliers, the plurality of students and the plurality of tutors; and (3) receive a set of solution tutorials that correspond to the set of challenging assessment questions from the at least one of the plurality of content-suppliers, the plurality of students and the plurality of tutors.

Additional aspects of the present disclosure contemplate the at least one processor is further configured to receive from, at least one of the plurality of content-suppliers, the plurality of students and the plurality of tutors, a similar question with regard to the challenging assessment question testing a similar aspect as the challenging assessment question.

Additional aspects of the present disclosure contemplate the at least one processor is further configured to notify one or more students who answered the challenging assessment question incorrectly to attempt the similar question after a predetermined time interval.

Additional aspects of the present disclosure contemplate the at least one processor is further configured to provide a machine-generated similar question with regard to the challenging assessment question testing a similar aspect as the challenging assessment question.

Additional aspects of the present disclosure contemplate the at least one processor is further configured to store a record of a number of incorrect attempts for each of the plurality of assessment questions.

Additional aspects of the present disclosure contemplate the at least one processor is further configured to identify the at least one assessment question of the plurality of assessment questions as a challenging assessment question by compiling a ranking list of incorrect attempts for the plurality of assessment questions so that the plurality of tutors create the solution tutorial based on the ranking list of incorrect attempts.

### V Exam Paper Based Method for Generating Education Material

Some students study for examination by attempting a past examination first. The method can be more efficient and effective especially for students in higher grades such as secondary school, junior colleges or universities. At higher level, some portions of the syllabus were already taught or learned outside the school. By attempting a past examination paper first, the student would know the topics that are more important, and the student will also understand the weak topics better. At such an age, not everything in the syllabus is worth spending the same amount of time. While the above are more for students from higher grades, the method can also be used for primary school students who are less than twelve years old. FIG. 10 shows a method 1000 for providing education materials. The method 1000 is based on the study method where the student starts doing past examination papers, or assessment tests that is set based on a similar format as the examination paper, or a plurality of special purpose assessment tests which is set specifically for this purpose. The special purpose assessment tests may be set such that each topic has one or more assessment questions from every topic. The method 1000 starts with the step of receiving one or more results from one or more assessment tests taken by the plurality of students. The one or more assessment tests comprises a plurality of assessment questions. As explained above, the one or more assessment tests may be a past examination paper, or a paper that is set based on a similar format as the examination paper, or a special purpose assessment tests set for the purpose of assessing areas where the plurality of students need education materials. The education materials comprises tutorials, lessons videos, additional practice questions that are set based on questions that many students do not know how to answer (challenging questions), a deliberate practice questions which comprises questions that are similar to challenging questions testing a similar aspect, a memo or notes on specific topic, or any other material that will help the students to understand the topic and gain knowledge so that the student will do well in the examination.

Next, the method 1000 proceeds to the step of compiling user-data of the plurality of assessment questions based on one or more results. The method 1000 also comprises the step of analyzing and compiling user-data. The user-data indicates how the plurality of students did with regard to the plurality of assessment questions. Each assessment question is linked to a topic of the syllabus. For example, the LMS 400 may generate a histogram showing statistics of students who answer each assessment question correctly. The statistics may be a percentage for each topic of the syllabus, or a percentage is grouped using various parameters for the tutors and the content suppliers to understand the needs of the students. The user-data may comprise first data taken from previous years of students, and second data taken from current year of students. Students from previous years are students who have taken the examination that the current years of students will take. The first data may also comprise an actual result of the previous years of student. Theoretically, students from every year should show the same trend or tendency but this may not be true because new education materials may be produced to address a specific area of the syllabus that the students are weak at. In addition, the syllabus may change. Therefore, user-data from previous years may not reflect data from current year. The word “previous year” and “current year” is used to illustrate students who had taken the examination and students who will be taking the examination and does not mean an actual calendar year that this specification is read. Compiling the user-data may comprise deducing the trend change by comparing the data from current year of students and the data from previous year of students. Alternatively, compiling user-data may comprise computing a trend or deduce areas where students need more education material by considering all students, or by considering students from current year without looking at previous years.

The method then proceeds to the step of communicating to a plurality of tutors so that the plurality of tutors will prepare at least one education material in response to the user data. The at least one education material in response to the user data may comprise a tutorial solution, a video lesson, or an additional study material that is related to an assessment question of the assessment test. In this way, the education materials will be created in response to the user-data, which reflects the needs of the students. The education materials will then be made available to the students through the LMS 400. Relying on the assessment tests alone to sieve assessment questions that the students are weak at may not be completely accurate because the students may guess correctly the answer. For this reason, the method 1000 further includes allowing the students a “not-sure” or “I don't know” option to mark, during taking the one or more assessment tests. The method may optionally comprise categorizing questions that more than a predetermined threshold number of students either answer incorrectly or marked as “not-sure” to be challenging questions. Next, the method 1000 may comprise communicating or providing feedback about the challenging questions to the tutors and/or content suppliers so that the tutors and/or content suppliers will consider creating an improved education material as compared to the education materials available to the plurality of students.

The data from the one or more assessment tests may be utilized to improve planning for each student. For example, based on the results from the one or more assessment tests, the LMS 400 may be able to analyze strength of a student for each of the topics in the syllabus. Topics that the student already did well should be allocated with less time. From the assessment test, the proficiency level-estimates of the students can be assessed more accurately as compared to a situation where no data are available, and one has to rely only on a progress information to revise the study plan as described in previous embodiments. For this purpose, the LMS 400 may be able to compute a customized user-data for each of the plurality of students, and subsequently generate a recommended study plan and a recommended set of study materials. The customized user-data tracks a time period each of the plurality of students spent within a specific time frame. The recommended study plan comprises a timetable for each of the plurality of students to complete a recommended set of study materials. The recommended set of study material is selected based on an estimation that each of the plurality of students will be able to complete the recommended set of study material within the specific time frame. For generating the recommended set of study material, the LMS 400 may compile a difficulty-level statistics for each of the plurality of assessment questions tracking number of the plurality of students who answer each of the plurality of assessment questions correctly. The LMS 400 may also compile a proficiency-level statistics for each of the plurality of students tracking a percentage number of the plurality of assessment questions that each of the plurality of students answers correctly. Next, the LMS 400 may compute a correlation mapping between the difficulty-level statistics and the proficiency-level statistics. Then the LMS 400 may select a customized compilation of the plurality of assessment questions for a first student of the plurality of students. The customized compilation of the plurality of assessment questions are selected from a portion of the plurality of assessment questions yet to be done by the first student. To improve the accuracy, the LMS 400 may adaptively update the difficulty-level statistics and the proficiency-level statistics so as to generate an updated customized compilation for the first student similar to the embodiments discussed previously.

Additional aspects of the present disclosure contemplate a method for providing an educational material for a plurality of students using an LMS, the method comprising: (1) receiving, from the plurality of students, one or more results, wherein the one or more results are from one or more assessment tests taken by the plurality of students, and wherein the one or more assessment tests comprises a plurality of assessment questions; (2) compiling user data of the plurality of assessment questions based on the one or more results, wherein the user data indicates how the plurality of students did with regard to the plurality of assessment questions; (3) communicating the user data to a plurality of tutors for the plurality of tutors to prepare at least one education material in response to the user data; and (4) making the at least one education material accessible, to the plurality of students, through the LMS.

Additional aspects of the present disclosure contemplate the user data comprises statistics on number of students who answer correctly each of the plurality of assessment questions.

Additional aspects of the present disclosure contemplate the method further comprising categorizing one or more of the plurality of assessment questions that a predetermined number of students answered incorrectly or marked as ‘not-sure’ as a challenging-question.

Additional aspects of the present disclosure contemplate the at least one education material in response to the user data comprises a solution tutorial or a video tutorial explaining how to answer the challenging-question.

Additional aspects of the present disclosure contemplate the at least one education material in response to the user data comprises an additional similar question that tests a similar aspect as the challenging-question.

Additional aspects of the present disclosure contemplate the method further comprising communicating the user data to a plurality of content-suppliers for the plurality of content-suppliers to prepare one additional content or a learning material related to the challenging-question in response to the user data.

Additional aspects of the present disclosure contemplate the method further comprising providing a video bridge or an audio bridge connecting one of the plurality of tutors and one of the plurality of students so that the one of the plurality of tutors can provide tuition on the challenging-question for the one of the plurality of students through the video bridge or the audio bridge.

Additional aspects of the present disclosure contemplate the method further comprising communicating the user data to a plurality of content-suppliers for the plurality of content-suppliers to prepare a new improved material after considering the user data.

Additional aspects of the present disclosure contemplate the method further comprising computing a customized user data for each of the plurality of students so as to generate a recommended study plan, wherein: (1) the customized user data tracks a time period each of the plurality of students spent within a specific time frame; (2) the recommended study plan comprises a time table for each of the plurality of students to complete a recommended set of study materials; and (3) the recommended set of study material is selected based on an estimation that each of the plurality of students will be able to complete the recommended set of study material within the specific time frame.

Additional aspects of the present disclosure contemplate the method further comprising: (1) compiling a difficulty-level statistics for each of the plurality of assessment questions tracking number of the plurality of students who answer each of the plurality of assessment questions correctly; and (2) compiling a proficiency-level statistics for each of the plurality of students tracking a percentage number of the plurality of assessment questions that each of the plurality of students answers correctly.

Additional aspects of the present disclosure contemplate the method further comprising: (1) computing a correlation mapping between the difficulty-level statistics and the proficiency-level statistics; and (2) selecting a customized compilation of the plurality of assessment questions for a first student of the plurality of students, wherein the customized compilation of the plurality of assessment questions are selected from a portion of the plurality of assessment questions yet to be done by the first student.

Additional aspects of the present disclosure contemplate the method further comprising periodically updating the difficulty-level statistics and the proficiency-level statistics so as to generate an updated customized compilation for the first student.

Additional aspects of the present disclosure contemplate an LMS for providing an educational material for a plurality of students using an LMS, the LMS comprising: at least one processor and computer memory coupled to the processor, wherein the computer memory comprises instructions that are executable by the processor, and wherein the instructions comprise: (1) a content database that comprises one or more assessment tests, wherein the one or more assessment tests comprise a plurality of assessment questions; (2) a learner interface component configured to let a plurality of students take the one or more assessment tests; (3) a user database that comprises one or more results from the one or more assessment tests taken by the plurality of students; (4) a prior use information processing component configured to compile a prior-use-data of the plurality of assessment questions based on the one or more results; (5) a prior use database for storing a prior use record of the prior-use-data, wherein the prior-use-data indicates how the plurality of students did with regard to each of the plurality of assessment questions; and (6) a tutor interface component for communicating at least a summary of the prior-use-data to a plurality of tutors so that the plurality of tutors will prepare at least one education material in response to the prior-use-data.

Additional aspects of the present disclosure contemplate a content administration component configured to identify one or more of the plurality of assessment questions that a predetermined number of students answered incorrectly or marked as ‘not-sure’ as a challenging-question.

Additional aspects of the present disclosure contemplate a content-supplier interface component for communicating the prior-use-data to a plurality of content-suppliers so that the plurality of content-suppliers prepare one additional content, or a learning material related to the challenging-question.

Additional aspects of the present disclosure contemplate (1) a study plan administration component for preparing a customized prior-use-data for each of the plurality of students so as to generate a recommended study plan, wherein the recommended study plan comprises a time table for each of the plurality of students to complete a recommended set of study materials; and (2) a scheduling component for tracking a time period where each of the plurality of students spent within a specific time frame intervals, wherein the recommended set of study material is selected such that each of the plurality of students will be able to complete the recommended set of study material within a specific time frame.

Additional aspects of the present disclosure contemplate (1) the content database comprises a difficulty-level statistics based on a number of the plurality of students who answer each of the plurality of assessment questions correctly; and (2) the user database comprises a proficiency-level statistics based on a percentage number that each of the plurality of students answers correctly the plurality of assessment questions.

Additional aspects of the present disclosure contemplate (1) a prior use information processing component configured to compute a correlation mapping between the difficulty-level statistics and the proficiency-level statistics; and (2) a content selection component configured to select a customized compilation of the plurality of assessment questions for a first student of the plurality of students, wherein the customized compilation of the plurality of assessment questions are selected from a portion of the plurality of assessment questions yet to be done by the first student.

Additional aspects of the present disclosure contemplate a content administration component, wherein the content administration component is configured to periodically update the difficulty-level statistics and the proficiency-level statistics so as to generate an updated customized compilation for the first student.

Additional aspects of the present disclosure contemplate a computer system for providing an LMS to a student, the computer system comprising a memory and at least one processor coupled to the memory, the at least one processor is configured to: (1) store a test result record comprising one or more assessment results, wherein the one or more assessment results is generated when a plurality of students took one or more assessment tests, and wherein the one or more assessment tests comprise a plurality of assessment questions; (2) generate user data of the plurality of assessment questions based on the one or more assessment results, wherein the user data indicates how the plurality of students did with regard to the plurality of assessment questions; (3) communicate the user data to a plurality of tutors for the plurality of tutors to prepare at least one education material in response to the user data; and (4) make available the at least one education material to the plurality of students.

Additional aspects of the present disclosure contemplate the at least one processor is further configured to receive an input from the plurality of students to identify a question as “not sure” for an assessment question that the plurality of students do not know how to answer.

Additional aspects of the present disclosure contemplate the user data comprises a record for each of the plurality of assessment questions that shows how many students answered correctly or made a “not sure” label on each of the plurality of assessment questions.

Additional aspects of the present disclosure contemplate the at least one education material comprises solution tutorials for assessment questions that more than a predetermined threshold of students answered incorrectly or marked as “not sure”.

Additional aspects of the present disclosure contemplate the at least one processor is further configured to compute a customized study plan for each of the plurality of students based on the user data, and wherein the customized study plan comprises a schedule for each of the plurality of students to complete a selected study material that is selected based on the user data.

### VI Exam Readiness Infographic Report

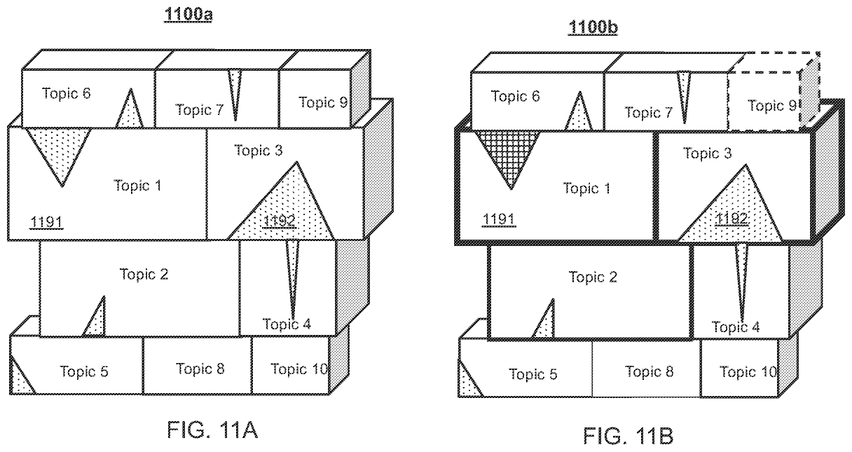

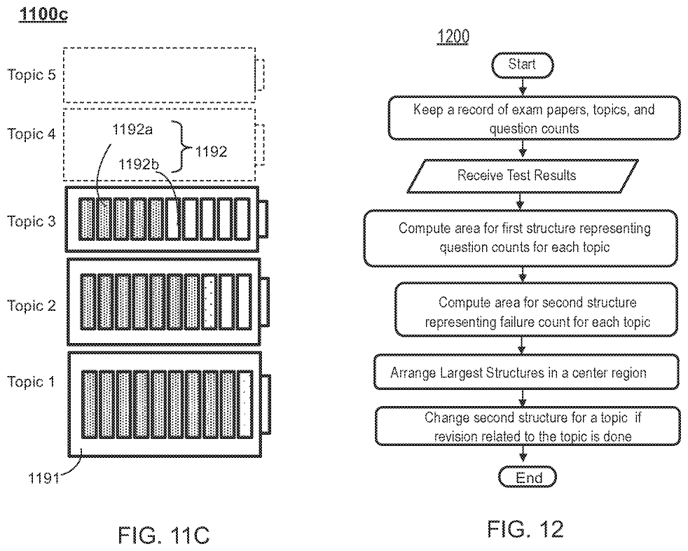

FIGS. 11A-11C show an apparatus for evaluating exam-readiness of a student. The apparatus may comprise an evaluation infographic reports 1100a-1100c that provides an indication of examination readiness of a student. Referring to FIG. 11A, the infographic report 1100a comprises a plurality of first graphic-structures 1191. Each of the first graphic-structures 1191 corresponds to a topic in the syllabus of an examination. Some topics may be more popular in the examination. In other words, the frequency for an examination question from those topics will be higher than other topics. The importance for each topic can be assessed based on past examination papers. For this reason, the first graphic-structures 1191 has a parameter that differs in accordance with the frequency of the topics to be included as an examination. Referring to FIG. 11A, the first graphic-structure 1191 of a specific topic has an area that correspond to the question count from the specific topic. By arranging the first graphic-structure in this manner, the student will have an idea regarding the importance of each topic. In the example shown in FIG. 11A, important topics are Topics 1-4 whereas Topics 9-10 are less important. The number of past-exam papers to compute the area for the first graphic structure 1191 may be determined by the student as his user preference input, or a parameter in the LMS 400 that is predetermined.

Before the examination, the student may sit for a trial examination or do practice test paper or past examination papers. The test results will then be input to the LMS 400 so as to generate failure record. The failure record comprises a plurality of failure rates computed for each of the plurality of topics. A failure rate for a specific topic may be computed by calculating a total number of questions that the student answered incorrectly for the specific topic as compared to the total number of questions from the specific topic. Each of the plurality of second graphic structures 1192 will have a size that corresponds to the failure rate of the specific topic. For example, if Topic 1 has a size of 2 cm² and the failure rate for Topic 1 is 10%, the area of the corresponding second graphic structure 1192 for Topic 1 is 0.2 cm². The corresponding second graphic structure 1192 may be represented as a gap, a void, a cracked region or a representation that shows the corresponding second graphic structure 1192 as a missing piece or a break-away piece of the corresponding first graphic structure 1192. For example, as shown in FIG. 11A, the first graphic structure is represented as a brick-like structure and the second graphic structure is represented as a cracked region inside the brick. In this way, the student will get an impression of this exam-readiness in a graphic form. Per the example shown in FIG. 11A, the student would understand that the revision for all topics is completed but there are major weakness in Topic 1 and Topic 3. Topics 8-10 are perfect, but Topics 2, 4, 5, 6, 7 could have been improved. The brick-like structure can be replaced with other forms showing a similar concept. For example, as shown in FIG. 11C, the first graphic structure 1191 comprises a battery like structure, and the second graphic structure 1192 comprises a representation of battery strength. The size of the battery 1191 comprises an indication of the importance of the topic. The size of the battery 1191 in FIG. 11C is shown as the width, but in another embodiment, the size may be represented by multiple-batteries of similar types. The second graphic structure 1192 is represented by the battery strength that indicates the failure rates. The battery strength, as shown in FIG. 11C forms an inner structure of the based building block of a battery. In general, the first graphic structure 1191 is shown as a base building block and the second graphic structure 1192 is shown as a missing piece, or an inner structure of the based building block.

Other parameters of the first graphic structure 1191 and the second graphic structure 1192 may be used to represent other aspect of the studies. For example, the thickness outline of the brick may be shown in accordance with the hours spent studying the topic as shown in FIG. 11B. In another embodiment where thickness of each brick is shown, the thickness of the brick may be shown to represent number of hours spent to study the corresponding topic. In FIG. 11C, the hours spent may be represented using a third parameter such as the color of the battery or the first graphic structure 1191. If a topic is not prepared at all, the specific topic may be represented as a missing first graphic structure such as using a dash line as shown in the first graphic-structure for Topic 9 in FIG. 11B. In FIG. 11C, if a topic is not prepared, the first graphic structure 1191 is represented as an empty battery slot. In another embodiment, the topic not prepared may be shown using a grey-out line or a lighter color (as compared to the line of the bricks) to create an impression of missing base structure. If action or steps had been taken to revise a specific topic, the second graphic structure 1192 may be shown using a different pattern as shown in the second graphic 1192 of Topic 1 in FIG. 11B. In this way, a user may have more graphical representation about his readiness for examination.

The infographic reports 1100a-1100c may be used as a tool that the student can rely on to prepare for an examination. For example, when the student starts to prepare the examination, the infographic report shows every first graphic-structures as a missing piece. The student will then spend time and effort to prepare for each topic. In the process, the first graphic-structures will be built up and become complete. In the embodiment shown in FIG. 11A-11B, the readiness is shown as in a form of a fortress wall. If everything is well prepared, and the students did well in the trial test paper, the wall is not penetrable and implies that the student is completely ready. However, inevitably, the students will be unlikely to get full marks in the trial exam, especially for the topics that the student is weak at. The student is then encouraged to “patch-up” the weak areas and get ready for the actual examination. In this way, the infographic report 1100a-1100c can show a quantifiable way to express exam-readiness. Optionally, the infographic reports 1100a-1100c may also serve as a progress report similar to the embodiment discussed in FIG. 1 and FIG. 2E. For example, the LMS 400 may define a minimum threshold for a student to complete studying one of the plurality of topics. The minimum threshold may be quantified in terms of numbers, or in terms of a minimum study material. When the student starts his studying, the plurality of first graphic-structure 1191 may be built up. The student will able to visualize the progress made towards the complete readiness.

In other words, the reports 1100A-1100c may form an apparatus for a student to evaluate readiness for taking an examination. The apparatus 1100a comprises a plurality of base graphic-structures 1191 representing a plurality of topics. The plurality of base graphic-structures are the base building blocks and may be shown as a brick-like structure as shown in FIG. 11A. The examination is set to examine the student on the plurality of topics wherein a topic from the plurality of topics has a question count that correlates to a total number of questions from the topic in a predetermined number of past exam papers. A first parameter of the plurality of base graphic-structures 1191 is presented in accordance with the question count for the topic. A plurality of secondary graphic-structures 1192 representing a plurality of failure rates of the student in one or more trial papers. A failure rate from the plurality of failure rate is a ratio number of questions the student answered incorrectly in the one or more trial papers as compared to the total number of questions for the topic in the one or more trial papers. A second parameter of the plurality of secondary graphic-structures 1192 is presented in accordance with the failure rate for the topic. For example, in a two-dimensional representation of the apparatus 1100a, the first parameter is the area of each of the plurality of base graphic-structures, and the second parameter is area of each of the plurality of secondary graphic-structures. As shown in FIGS. 11A-11C, the plurality of secondary graphic-structures 1192 are optionally presented within the plurality of base graphic-structures 1191. The plurality of secondary graphic-structures are presented as missing pieces of the plurality of base graphic-structures. In addition, a corresponding base graphic structure of the plurality of base graphic-structures for a topic is presented as a missing-base graphic-structure if the student has not studied for the topic. For example, as shown in FIG. 11B, the missing-base graphic-structure 119 is presented in dotted line. In FIG. 11C, the missing-base graphic-structure comprises an empty battery slot. In another embodiment, the missing base graphic-structure may be presented in a lighter color as compared to the plurality of base graphic-structures to produce the missing effect. In this way, the readiness of the student for the examination may be visually represented as the area of the wall. If the student has studied for all topics, the apparatus shows a complete wall. However, if the student has a weakness in a specific topic, the corresponding base graphic-structure will have a missing part that prompt the student to take action. As shown in FIG. 11B, the plurality of base graphic-structures has an additional parameter presented in accordance with the first number of hours which is the line of the base graphic structure which is drawn in accordance with the total number of hours the student has studied. In FIG. 11C, the number of hours spent may be represented by showing different colors. For example, a darker color for more hours spent.

FIG. 12 shows a method 1200 for evaluating examination readiness as discussed in FIGS. 11A-11C. Referring to FIG. 12, the LMS 400 may store a record of one or more past exam papers, a plurality of questions in the one or more exam papers, a syllabus that defines a plurality of topics and question counts for each topic. Optionally, the student may provide an input as to how many past-examination papers the LMS 400 needs to consider when generating the evaluation infographic report. In theory, one examination paper is sufficient, but generally, three to five past examination papers would provide a more representative report. For some examinations where there are less questions in the examination paper, the number of past examination papers that the LMS has to consider will increase. Next, the method 1200 proceeds to the step of receiving test results from the student. The test results comprise trial examination paper results or any other pre-test results. Next, the method proceeds to compute area for the plurality of first structures by making a corresponding first graphic-structure for a corresponding topic a size that corresponds with the count of questions from the corresponding topic. The method 1200 then computes the area for the second graphic structure such that a ratio of the area of a corresponding second graphic structure relative to a corresponding first graphic structure for a corresponding topic reflects the failure rate of the students in the test results for the corresponding topic. The method 1200 then proceeds to generate the first and second graphic-structures 1191, 1192 in a display such that larger first graphic-structures 1191, 1192 is located at a center region of the screen. Next, if a revision has been done for a corresponding topic, the corresponding second structure for the topic may be changed to indicate that a remedial step has been taken. While a sequential step is illustrated, the sequence for each step may be changed and may not be followed strictly. For example, the first graphic structure may be computed or generated before the test results are received. In yet another example, the area of the first and second graphic-structures 1191, 1192 may be calculated while generating and arranging the first and second graphic-structures 1191, 1192 in a display, and not prior to the creation of the images.

Additional aspects of the present disclosure contemplate a method for evaluating readiness of a student in taking an examination, the method comprising (1) storing an electronic examination record in computer memory, wherein the electronic examination record comprises a description of one or more exam papers that includes a plurality of questions testing the student on a plurality of topics; (2) computing a count record that comprises a plurality of question counts for the plurality of topics, and wherein a corresponding question count correlates with a total number of questions for a topic in the electronic examination record; (3) receiving one or more test results from the student based on one or more trial tests; (4) computing a failure record, wherein the failure record comprises a plurality of failure rates for the plurality of topics, wherein a corresponding failure-rate comprises a ratio representing number of questions the student answered incorrectly as compared to a total number of questions in the one or more test results for the topic; (5) displaying, to the student, a plurality of first graphic-structures that correspond to the plurality of topics, respectively, wherein a first graphic-structure from the plurality of first graphic-structures comprises a first area that correlates with the corresponding question count for the topic; and (6) displaying, to the student, a plurality of second graphic-structures, wherein a second graphic-structure from the plurality of second graphic-structures comprises a second area that correlates with the corresponding failure-rate for the topic.

Additional aspects of the present disclosure contemplate the method further comprising arranging the plurality of first graphic-structures on a screen such that a graphic-structure having a larger area is located at a center portion of the screen.

Additional aspects of the present disclosure contemplate the method further comprising: (1) receiving a first progress information from the student, wherein the first progress information comprises information related to whether the student has studied the topic; and (2) representing a corresponding first graphic-structure of the plurality of first graphic-structures as a missing graphic-structure for a corresponding topic if the student has not studied the corresponding topic.

Additional aspects of the present disclosure contemplate the method further comprising: (1) receiving a second progress information from the student, wherein the second progress information comprises information related to a quantity of time the student spent on each of the plurality of topics; and (2) representing a parameter of the plurality of first graphic-structures in accordance with the quantity of time the student spent.

Additional aspects of the present disclosure contemplate each of the plurality of first graphic-structures is shown in a solid line having a thickness that is in accordance with the quantity of time the student spent.

Additional aspects of the present disclosure contemplate the method further comprising displaying the plurality of second graphic-structures as a plurality of missing pieces inside the plurality of first graphic-structures.

Additional aspects of the present disclosure contemplate the method further comprising displaying a first missing piece of the plurality of missing pieces for a first topic as a filled up area to differentiate from the plurality of first graphic-structures and the plurality of second graphic-structures when revision is completed for the first topic.

Additional aspects of the present disclosure contemplate the method further comprising representing the plurality of first graphic-structures as a plurality of brick-like graphic-structures.

Additional aspects of the present disclosure contemplate the method further comprising representing each of the plurality of second graphic-structures as a crack-like structure, a gap, or a void within the plurality of brick-like graphic-structures.

Additional aspects of the present disclosure contemplate the method further comprising representing the plurality of brick-like graphic-structures as a wall-like graphic-structure by joining up the plurality of brick-like graphic-structures.

Additional aspects of the present disclosure contemplate the method further comprising representing the plurality of first graphic-structures as a plurality of battery-like graphic-structures.

Additional aspects of the present disclosure contemplate the method further comprising representing each of the plurality of second graphic-structures as a battery-strength indicating structure disposed within the plurality of the first graphic-structures.

Additional aspects of the present disclosure contemplate a method for evaluating readiness of a student in taking an examination, the method comprising (1) storing a syllabus record, wherein the syllabus record comprises a plurality of topics; (2) storing an examination record, wherein the examination record comprises a description of one or more exam papers which includes a plurality of questions testing the student on the plurality of topics; (3) computing a question count for a topic, wherein the question count includes a number of questions from the topic; (4) receiving one or more test results from the student; (5) computing a failure-rate, wherein the failure-rate includes a ratio representing a number of questions the student answered incorrectly in the topic as compared to a total number of questions of the topic; (6) displaying, to the student, a first graphic-structure from a plurality of first-structures, wherein the first graphic-structure has a first area that correlates with the question count; and (7) displaying, to the student, a second graphic-structure, wherein the second graphic-structure has a second area that correlates with the failure-rate.

Additional aspects of the present disclosure contemplate a computer system for assisting a student to evaluate examination preparation, the computer system comprising a memory and at least one processor coupled to the memory, the at least one processor is configured to: (1) store an electronic examination record for one or more examination papers, wherein the one or more examination papers comprises a plurality of questions testing the student on a plurality of topics; (2) store a count record, wherein the count record comprises a plurality of question counts for the plurality of topics, and wherein a question count from the plurality of question counts correlates with a total number of questions for a topic in the electronic examination record; (3) receive one or more test results from the student after the student takes one or more trial tests; (4) generate a failure rate record, wherein the failure rate record comprises a failure rate for the plurality of topics, wherein a corresponding failure rate correlates with a percentage of questions in the one or more test results that the student answered incorrectly in the topic; and (5) display a readiness report to the student, wherein the readiness report comprises: (a) a plurality of first graphic-structures representing the plurality of topics respectively, wherein a first graphic-structure from the plurality of first graphic-structures has a first area that correlates with the question count for the topic; and (b) a plurality of second graphic-structures, wherein a second graphic-structure from the plurality of second graphic-structures has a second area that correlates with the failure rate of the topic respectively.

Additional aspects of the present disclosure contemplate the at least one processor is further configured to display the plurality of first graphic-structures and the plurality of second graphic-structures such that the plurality of second graphic-structures are placed inside the plurality of first graphic-structures respectively.

Additional aspects of the present disclosure contemplate the at least one processor is further configured to display the plurality of first graphic-structures and the plurality of second graphic-structures such that the plurality of second graphic-structures are represented as missing pieces of the plurality of second graphic-structures.

Additional aspects of the present disclosure contemplate the at least one processor is further configured to display each of the plurality of first graphic-structures as a brick-like structure, and each of the plurality of second graphic-structures is represented as a crack or a void of the brick-like structure.

Additional aspects of the present disclosure contemplate the at least one processor is further configured to display each of the plurality of first graphic-structures as a basic building block, and each of the plurality of second graphic-structures as a missing piece of the basic building block.

Additional aspects of the present disclosure contemplate the at least one processor is further configured to receive a further input from the student regarding a remedial revision for a revised topic of the plurality of topics, and wherein a corresponding second structure for the revised topic is represented differently to represent the remedial revision.

Additional aspects of the present disclosure contemplate the plurality of first graphic-structures are presented sequentially in accordance with a progress of the student for each of the plurality of topics.

Additional aspects of the present disclosure contemplate the at least one processor is further configured to receive a preparation input that comprises a number of hours spent by the student for each of the plurality of topics.

Additional aspects of the present disclosure contemplate the at least one processor is further configured to present the plurality of first graphic-structures such that a parameter of the plurality of first graphic-structures is in accordance with the number of hours spent.

Additional aspects of the present disclosure contemplate an LMS for evaluating readiness of a student for an examination, the LMS comprising: at least one processor and computer memory coupled to the at least one processor, wherein the computer memory comprises instructions that are executable by the at least one processor, and wherein the instructions comprise: (1) a syllabus database component comprising one or more past examination papers, and a plurality of topics, wherein prior students are examined on the plurality of topics in the one or more past examination papers; (2) a statistic record component comprising a plurality of question counts, wherein a corresponding question count from the plurality of question counts for a topic represent a total number of question counts from the topic in the one or more past examination papers; (3) a student interface component for receiving a test-paper results from the students after taking one or more trial tests; (4) an assessment test management component configured to generate a plurality of failure rates for the plurality of topics, wherein a corresponding failure rate from the plurality of failure rate for the topic correlates with a ratio of number of questions that the student answered incorrectly in the test-paper results as compared to a total number of questions for the topic in the test-paper results; and (5) a progress monitoring component for generating a graphic report to the student, wherein the graphic report comprises: (a) a plurality of base graphic-structures to represent readiness for the plurality of topics, wherein each of the plurality of base graphic-structures has a first parameter presented in accordance with the plurality of question counts for the plurality of topics; and (b) a plurality of secondary graphic-structures presented within the plurality of base graphic-structures to represent weakness in the plurality of topics, wherein each of the plurality of secondary graphic-structures has a second parameter presented in accordance with the plurality of failure rates of the plurality of topics.

Additional aspects of the present disclosure contemplate the plurality of base graphic-structures are presented such that an area of each of the plurality of base graphic-structures is selected as the first parameter.

Additional aspects of the present disclosure contemplate the plurality of secondary graphic-structures are presented such that an area of each of the plurality of secondary graphic-structures is selected as the second parameter.

Additional aspects of the present disclosure contemplate each of the plurality of base graphic-structures are presented as a brick-like structure, and wherein each of the plurality of secondary graphic-structures are presented as a crack or a void area inside the brick-like structure.

Additional aspects of the present disclosure contemplate an apparatus for a student to evaluate readiness for taking an examination, the apparatus comprising: (1) a plurality of base graphic-structures representing a plurality of topics, wherein the examination is set to examine the student on the plurality of topics wherein a topic from the plurality of topics has a question count that correlates to a total number of questions from the topic in a predetermined number of past exam papers; (2) a first parameter of the plurality of base graphic-structures presented in accordance with the question count for the topic; (3) a plurality of secondary graphic-structures representing a plurality of failure rates of the student in one or more trial papers, wherein a failure rate from the plurality of failure rate is a ratio number of questions the student answered incorrectly in the one or more trial papers as compared to the total number of questions for the topic in the one or more trial papers; and (4) a second parameter of the plurality of secondary graphic-structures presented in accordance with the failure rate for the topic.

Additional aspects of the present disclosure contemplate the plurality of base graphic-structures are presented in a two-dimensional form, and wherein an area of each of the plurality of base graphic-structures is selected as the first parameter.

Additional aspects of the present disclosure contemplate the plurality of base graphic-structures are presented in a two-dimensional form, and wherein an area of each of the plurality of secondary graphic-structures is selected as the second parameter.

Additional aspects of the present disclosure contemplate the plurality of secondary graphic-structures are presented within the plurality of base graphic-structures.

Additional aspects of the present disclosure contemplate the plurality of secondary graphic-structures are presented as missing pieces of the plurality of base graphic-structures.

Additional aspects of the present disclosure contemplate a corresponding base graphic structure of the plurality of base graphic-structures for a topic is presented as a missing-base graphic-structure if the student has not studied for the topic.

Additional aspects of the present disclosure contemplate the missing-base graphic-structure is presented in dotted line or a lighter color as compared to the plurality of base graphic-structures.

Additional aspects of the present disclosure contemplate the student studied for a first number of hours for a first topic from the plurality of topics, and wherein the plurality of base graphic-structures has an additional parameter presented in accordance with the first number of hours.

Although specific embodiments of the invention have been described and illustrated herein above, the invention should not be limited to any specific forms or arrangements of parts so described and illustrated. For example, the steps in each of the method step may not follow the exact sequence as illustrated but may include other possible sequences as understood by a person skilled in the art. The specification was prepared such that a specific embodiment focuses on one aspect or one feature and may be silent as to the other aspect or features. Therefore, a feature explained in a specific embodiment may be combined into another embodiment although the specification does not explicitly express such embodiment. Unless specifically mentioned otherwise, any feature discussed in a specific embodiment or a sub-heading shall be freely combined with other embodiments. The scope of the invention is to be defined by the claims appended hereto and their equivalents. Throughout the specification and the claim, the terms first, second and third may be used as identifiers only. For example, the term “second number” does not mean that there is another “first number”, but the term “second number” is introduced to differentiate a number (second number) from another number (first number).

We claim:

1. A method for providing a customized study content to a student, the method comprising:
providing an LMS for maintaining a record of a content proposal, wherein the content proposal comprises at least one of a plurality of study materials and a plurality of assessment questions;
determining, with the LMS, a plurality of content-related-estimates, wherein the plurality of content-related-estimates comprise at least one of a difficulty level-estimate and a study time-estimate for the content proposal;
determining, with the LMS, a plurality of user-related-estimates, wherein the plurality of user-related-estimates comprises at least one of a proficiency level-estimate and an available time-estimate for the student;
determining a selected portion of the content proposal as the customized study content for the student; and
adaptively adjusting at least one of the plurality of content-related-estimates and the plurality of user-related-estimates.

2. The method of claim 1, wherein the selected portion of the content proposal are selected as the customized study content for the student if the study time-estimate of the selected portion of the content proposal is less than the available time-estimate.

3. The method of claim 1, further comprising adaptively adjusting at least one of the difficulty level-estimate and the study time-estimate of the plurality of study materials.

4. The method of claim 3, wherein:
a first content of the content proposal is provided to a group of students, wherein the first content comprises a first content assessment question;
a first number of the group of students, who have lower proficiency level-estimates compared to the difficulty level-estimate of the first content, answer correctly the first content assessment question; and
the difficulty level-estimate of the first content is adjusted lower, when the first number is more than a first predetermined threshold.

5. The method of claim 3, wherein:
a second content of the content proposal is provided to a group of students, wherein the second content comprises a second content assessment question;
a second number of the group of students, who have higher proficiency level-estimates compared to the difficulty level-estimate of the second content, answer incorrectly the second content assessment question; and
the difficulty level-estimate of the second content is adjusted higher when the second number is higher than a second predetermined threshold.

6. The method of claim 3, wherein:
a third content of the plurality of study materials is provided to a group of students; and
the study time-estimate is adjusted when more than a predetermined percentage of the group of students completed faster or slower than the study time-estimate of the third content by a predetermined margin.

7. The method of claim 1, further comprising adaptively adjusting at least one of the proficiency level-estimate and the available time-estimate of the student.

8. The method of claim 1, wherein the proficiency level-estimate is adjusted higher when the student answers correctly a predetermined amount of questions with a difficulty level-estimate higher than the proficiency level-estimate of the student.

9. The method of claim 1, further comprising collecting a health-related information about the student; and adjusting the available time-estimate for the student based on the health-related information.

10. The method of claim 1, further comprising providing a practice test paper selected from the customized study content, wherein:
a first predetermined portion of the practice test paper is selected such that the difficulty level-estimate of the first predetermined portion of the practice test paper is higher than the proficiency level-estimate of the student; and
a second predetermined portion of the practice test paper is selected such that the difficulty level-estimate of the first predetermined portion of the practice test paper is lower than the proficiency level-estimate of the student.

11. The method of claim 10, wherein a ratio of the first predetermined portion and the second predetermined portion is adjusted in accordance with a personality input about the student that is related to self-esteem of the student.

12. The method of claim 10, wherein a substantial portion of the practice test paper is selected such that the proficiency level-estimate of the student is substantially equal to the difficulty level-estimate of the substantial portion of the practice test paper.

13. The method of claim 10, wherein the first predetermined portion and the second predetermined portion are adaptively selected such that the student scores in a range of 50%-80% in the practice test paper.

14. The method of claim 10, further comprising:
obtaining an anxiety level information of the student; and
selecting, by using the LMS, the first predetermined portion and the second predetermined portion in accordance with the anxiety level information.

15. The method of claim 10, further comprising:
providing a progress projection; and
generating an actual progress information based on a percentage of the customized study content completed by the student,
wherein a ratio composition of the first predetermined portion and the second predetermined portion is adjusted based on a comparison between the progress projection and the actual progress information.

16. A learning management system (LMS) for providing a customized study content to a student, the LMS comprising:
at least one processor; and
computer memory coupled to the at least one processor, wherein the computer memory comprises instructions that are executable by the at least one processor, and wherein the instructions comprise:
a content proposal record, wherein the content proposal record comprises at least one of a plurality of study materials and a plurality of assessment questions;
a content admin component configured to determine a plurality of content-related-estimates for the content proposal record, wherein the plurality of content-related-estimates comprise at least one of a difficulty level-estimate and a study time-estimate;
a user management component configured to determine a plurality of user-related-estimates for the student, wherein the plurality of user-related-estimates comprises at least one of a proficiency level-estimate and an available time-estimate for the student; and
a study plan admin component configured to determine a selected portion of the content proposal record as the customized study content for the student, wherein at least one of the plurality of content-related-estimates and the plurality of user-related-estimates are configured to be adaptively adjusted through one of the content admin component and the user management component respectively.

17. The LMS of claim 16, wherein the instructions further comprise a welfare management component configured to collect a health-related information, and wherein the user management component is configured to adjust the available time-estimate for the student based on the health-related information.

18. The LMS of claim 17, wherein the welfare management component is coupled to a pulse rate tracking device that tracks the pulse rate of the student, and the welfare management component is configured to determine whether the student is in an anxious state based on the pulse rate.

19. The LMS of claim 17, wherein the welfare management component is configured to receive a confidence-level-input about the student from a coach or a tutor of the student, and wherein the instructions further comprises an assessment test management component configured to generate an assessment test for the student, and wherein the assessment test has a difficulty level that correlates with the confidence-level-input.

20. A method for managing learning of a student, comprising:
receiving user inputs related to the learning, the user inputs including one or more subjects that is targeted by the student, one or more study materials related to the one or more subjects, a targeted learning period and an available study time;
generating a study plan comprising an estimation of identified study material and an estimation of time needed to complete the identified study material, wherein the identified study material comprises a subset of the one or more study materials;
receiving further user inputs related to a progress of the learning, the further user inputs including portions of the one or more study materials completed by the student;
identifying an outstanding content portion of the one or more study materials based on the further user inputs;
computing an estimation of time needed to complete the outstanding content portion for feasibility check;
computing a remaining time that corresponds to an amount of time remaining towards an end date of the targeted learning period; and
adaptively performing a feasibility check based on the remaining time and the estimation of time needed to complete the outstanding content portion.
